(19) United States
(12) Patent Application Publication
(10) Pub. No.: US 2023/0062593 A1
(43) Pub. Date: Mar. 2, 2023

(54) A SYNTHETIC COMPOSITE AS BONE GRAFT AND THE METHOD THEREOF

(71) Applicant: BONE SUBSTITUTES, Madurai, Tamil Nadu (IN)

(72) Inventor: Sankaralingam PUGALANTHI PANDIAN, Madurai, Tamil Nadu (IN)

(73) Assignee: BONE SUBSTITUTES, Madurai, Tamil Nadu (IN)

(21) Appl. No.: 17/792,940

(22) PCT Filed: Feb. 25, 2021
(86) PCT No.: PCT/IN2021/050184

§ 371 Date: Jul. 14, 2022

(30) Foreign Application Priority Data
Feb. 26, 2020 (IN) 202041008048

(51) Int. Cl. A61L 27/46; A61L 27/56; B33Y 80/00; B29C 67/20

(52) U.S. Cl. CPC A61L 27/46; A61L 27/56; B33Y 80/00; B29C 67/202; A61L 2430/02; B29K 2509/08

(57) ABSTRACT

The invention is for a synthetic composite for a bone graft comprising of: bio inert polymers comprising poly lactic acid, poly D, L-Lactic acid; bio active polymer consisting of polypropylene fumarate or diester of fumaric acid and propylene diol (1,2-Diol); and a bioactive inorganic component consisting of a metal fluorophosphates glass powder wherein the amount of the bioactive components is upto 30% (w/w) of the composite. The bioactive inorganic metal fluorophosphates glass powder of the composite is one of zinc fluorophosphate, magnesium fluorophosphate or silver fluorophosphate. The invention pertains to the method of making the scaffold, and also the 3D printed scaffold.

### FIELD OF INVENTION

The present invention pertains to the field of composites. Specifically, the invention pertains to the composites of bio-degradable polymers and bioactive fluorophosphate glass as synthetic bone graft, in the form of a powder or a scaffold and the method of making the same.

### BACKGROUND OF THE INVENTION

The quantity and the type of graft needed by the clinician depends upon the clinical condition and the situation. A surgeon who empties a bone cyst will need a lot of graft to fill the void and in doing so may need a granular filler so that all the nooks and corner is filled up. When the surgeon faces a case on non-union of fracture of a long bone, to bring in bridging callus, apart from good fixation of the fracture he will need to do shingling of the fracture ends to bring out and expose the mesenchymal cells to the exterior and over that lay down strips of iliac bone graft (phemister grafts) to induce and conduct bone union across the fracture site

When a compound fracture with bone loss is encountered, a cortical lesion demands excision of a segment of long bone, then the reconstruction needs a load bearing bone graft which will substitute the lost bone and biologically get converted to bone in a short duration of time without prolonging the morbidity of the patient.

When the metaphyseal end of the bone is diseased and needs removal, without amputating the limb salvage procedures are done with custom made prosthesis. Because the prosthesis has a specific life and can go for fatigue fracture, a custom made graft implant made by rapid prototyping which after implantation will get converted to bone is a boon to the patient than the existing method.

Need of synthetic bone is increasing as the incidence of high speed accidents are increasing and salvage surgeries for bony lesions and tumours are increasing, while the hands of the surgeon are bound by the availability of auto graft because of their limited resource and the morbidity associated with reefing the graft from another site making another one incision. Allograft or bank bone always has the risk of disease transmission.

The material that was put to use initially was ceramics-hydroxyl apatite and tricalcium phosphate as bone graft substitutes. Hydroxyl apatite was only osteo-conductive and rarely was converted to bone even after years. It was not useful in replacing weight bearing function. Tri-calcium phosphate had minimal osteo-inductive capacity along with osteo-conduction but had no bio-conversion capability.

To have the advantage of bio-conversion certain specific bone hormones like “Bone Morphogenic Principle” simply called as BMP came into use. Like the same “Demineralised Bone Matrix (DMB)” was also marketed as bone graft substitute. The essential problem in their use is the phenomenal cost involved and they had good osteo-induction but were not good osteo-conductors. HENCH came out with the 45S5 glass which was a breakthrough as it was made from cheap chemicals, were osteo-conductive as well as osteo-inductive, was able to merge with the natural bone and is commercially available. The drawback with 45S5 glass is their very slow resorption, the longer time taken for bio-conversion, and their inability to be used as a weight bearing implant. To circumvent these problems, silica free phosphate bioglass and metal oxide doped bioglass came into the field.

Standardisation of the ideal mole percentage of fluoride resulted in the invention of fluorophosphates glasses which are much more bio active than the phosphate and silica glasses and had a higher rate of bioconversion. Doping them with metal oxides improved their physical properties and brought the elastic moduli close to that of the human bone. Scaffolding the fluorophosphate glass was essential to bring the molecule for clinical use.

Thus, there is a need in the field for a synthetic composite which is biocompatible and has several other characteristics such as being bioactive, biodegradable; nontoxic to the recipient; bio conductive; undergoes bioconversion, workable to the desired shape and cost effective

### OBJECT OF INVENTION

The object of the invention is for synthetic composites of bio inert polymers comprising of poly lactic acid, poly D, L-Lactic acid and bio active polymers consisting of polypropylene fumarate, diester of fumaric acid and propylene diol (1,2 Diol) and a bioactive inorganic component consisting of a metal fluorophosphates glass powder.

Another object of the invention is for granules, scaffolds like strips, cylinders and any other shape of the composites and the method of making the same.

Another object of the invention is for a scaffold preparation by 3 D printing.

### SUMMARY OF INVENTION

The invention is for a synthetic composite for a bone graft comprising of: bio inert polymers comprising poly lactic acid, poly D, L-Lactic acid; bio active polymer consisting of polypropylene fumarate or diester of fumaric acid and propylene diol (1,2-Diol); and a bioactive inorganic component consisting of a metal fluorophosphates glass powder wherein the amount of the bioactive components is upto 30% (w/w) of the composite.

In an aspect of the invention the bioactive inorganic metal fluorophosphates glass powder of the composite is one of zinc fluorophosphate, magnesium fluorophosphate or silver fluorophosphate.

In an aspect, the polylactic acid in the composite is in the range of 54% (w/w) to 68% (w/w); poly D, L-lactic acid is in the range of 10% (w/w) to 28% (w/w); 1,2 diol is in the range of 3% (w/w) to 10% (w/w); polypropylene fumarate is in the range of 3% (w/w) to 10% (w/w); the inorganic metal fluorophosphates glass powder in the composite is in the range of 10% (w/w) to 30% (w/w).

In an embodiment the composite is in the form of a powder, or a scaffold. The scaffold is a strip or a cylinder or a tube and the like as and when fabricated.

In an embodiment the synthetic composite of the invention is prepared by the following method comprising the steps of: mixing the composite in a solvent with the magnetic stirrer or sonicated to obtain a homogenous mixture; the mixture is casted over hot glass plate and the solution is brought to boil; and evaporation of the solvent by continuous boiling; and an interconnected porous scaffold with the homogenous distribution of the components of the composite was obtained.

The porosity of the scaffold ranges from 20%-40%.

In addition, the scaffold is also made of desired shape and desired porosity by a custom-made 3D printer by the Direct Ink Printing Technique.

The method of obtaining a scaffold by a custom-made 3D printer by the Direct Ink Printing Technique comprising the following steps: The components of the composites are homogenised and cooled to 10-30° C. The chamber temperature is kept at 30-40° C. The ink is loaded into a pressure-controlled, non adherent extruder. The input writing details were fed to the printer through the microchip. The nozzle diameter was fixed as 300 μm for easy extrusion. The volumetric flow rate was set at 5 mm³/s. The space between the lines of writing was 200 microns and the number of layers are designed 10. The desired shape, thickness, porosity, layers fed by a computer is written on a hot plate (100° C.). The movement of the hot plate in x, y, z direction were also pre-set and the commands was transferred by the microchip.

### BRIEF DESCRIPTION OF DRAWINGS AND FIGURES

FIG. 1 is bar diagram depicting the ALP activity of the dissolution products of metal oxide doped FP glasses at various concentrations of the glass.

FIGS. 2a and 2b. Thermal studies of the FP and MgFP glass powder.

FIGS. 2c and 2d. Thermal studies of the ZnFP and TiFPglass powder.

FIGS. 2e and 2f. Thermal studies of the ZrFP and AgFPglass powder.

FIG. 2g. Thermal studies of the SrFPglass powder.

FIG. 3 represents the FTIR spectrum of PPF.

FIG. 4. represents the DSC study of the PPF and FIG. 4a. represent the transition glass temperature of the PPF.

FIG. 5. FTIR spectrum of the Fumaric acid 1,2 propane diol.

FIG. 6. TG study of the Fumaric acid 1,2 propane diol.

FIG. 7. Characterization and Thermal analysis of the PLA.

FIG. 8. Characterization and Thermal analysis of the PDLLA.

FIG. 9. Preparation of flurophosphate glass by quenching from 1200 degrees to instant −170° C.

FIG. 10. Attempts of scaffolding by varying the concentration of polymers.

FIG. 11. Scaffolding attempts by varying the concentration of FP salts.

FIG. 12. Cell adhesion studies of the scaffold with relation to the variation in the components.

FIG. 13. Cell adhesion studies of the composites in relation to the variation in the components and the presence or absence of porosity.

FIG. 14. Different scaffolds achived by different methods of scaffolding.

FIG. 14a. SEM image of the scaffold made by Gel compression.

FIG. 14b. Preparation of scaffold by gel foam casting under rapid heating.

FIG. 15. Cytotoxicity (MTT) Assay of Endothelial Cells of scaffolds.

FIG. 15a. Cytotoxicity (MTT) Assay on Endothelial Cells of scaffolds (photomicrograph).

FIG. 16. RT-PCR Collagen II expression of the AgFP and ZnFP based scaffolds.

FIG. 17. RT-PCR Osteocalcin expression of the AgFP and ZnFP based scaffolds.

FIG. 18. RT-PCR Collagen II and Osteocalcin expression of Mg based scaffolds.

FIG. 19. RT_PCR RUN_X2 expression of scaffolds.

FIG. 20. Chondroitin sulphate levels of scaffolds' expression in SaOS2 cell lines.

FIG. 21. FTIR Spectra of the PPF based scaffolds (In-vitro evaluation—pre and post immersion).

FIG. 21a. Interpretation of the PPF based scaffolds (In-vitro evaluation—pre and post immersion).

FIG. 22. FTIR Spectra of the Diol based scaffolds (In-vitro evaluation—pre and post immersion).

FIG. 22a. Interpretaion of the Diol based scaffolds (In-vitro evaluation—pre and post immersion).

FIG. 23. FTIR Spectra of the MgFP based scaffolds (In-vitro evaluation—pre and post immersion).

FIG. 23a. Interpretaion of the MgFP based scaffolds (In-vitro evaluation—pre and post immersion).

FIG. 24. FTIR Spectra of the scaffolds of AgFP, ZnFP, MgFP (scaffolded by gel foam casting under rapid heating) (In-vitro evaluation—pre and post immersion).

FIG. 24a. Interpretation of the scaffolds of AgFP, ZnFP, MgFP (scaffolded by gel foam casting under rapid heating) (In-vitro evaluation—pre and post immersion).

FIG. 25. FTIR Spectra of the strip scaffold (in vitro evaluation—Pre and Post immersion).

FIG. 25a. Interpretaion of the strip scaffold (in vitro evaluation—Pre and Post immersion).

FIG. 26. FTIR Spectra of the cylindrical scaffold (in-vitro evaluation-Pre and Post immersion).

FIG. 26a. Interpretation of the cylindrical scaffold (in vitro evaluation-Pre and Post immersion).

FIG. 27. Photograph of strip and cylindrical scaffold made by gel foam casting under rapid heating.

FIG. 28. SEM micrograph of the Pre and Post immersion scaffold in two different magnification.

FIG. 28a. Depth of crystallisation (from both superior and inferior surface)—inner zone of the scaffold evaluated by SEM.

FIG. 29. Micro CT evaluation of the pre in-vitro of the cylindrical sample

FIG. 29a. Micro CT evaluation of the post in-vitro of the cylindrical sample

FIG. 30. SEM images of the cylindrical scaffold (pre immersion)

FIG. 30a. SEM images of the cylindrical scaffold (post immersion)

FIG. 30b. EDAX of the specimens pre and post in vitro evaluation.

FIG. 31 SEM image of a strip of scaffold (pre immersion and post immersion).

FIG. 32. Animal study to assess the efficacy of the granules of the scaffold.

FIG. 33. Post-operative X-ray of the femur bone.

FIG. 34. X-Ray of the Dissected specimen.

FIG. 35. Segment of the specimen studied in the HPE.

FIG. 36. Histo pathological evaluation of the specimen (EH stain and von kossa stain)

FIG. 37-a-b-c-d. Modified Tetrachrome staining of the specimen

FIG. 38. Animal study to assess the efficacy of Strips of the composites.

FIG. 39,39a,39b. Day 0 & Day 1, Day 9, Day 15 x-rays of the three animals (A, B, C AgFP, ZnFP, MgFP respectively).

FIG. 40, 40a.40b. CT. scan on day 19 of all three animals. (AgFP, ZnFP, MgFP respectively).

FIG. 41. Photographs of the dissected specimens (AgFP, ZnFP, MgFP respectively).

FIG. 42. X-Ray of the dissected specimens (AgFP, ZnFP, MgFP respectively).

FIG. 43 a,b,c,d. Histo pathological evaluation of the specimens (EH and Masson Trichrome stain).

FIG. 44 a,b,c,d. Histo pathological evaluation of the specimens by Modified Tetrachrome stain.

FIG. 45 (a) The control panel of the designed 3D printer.

FIG. 45(b) The pressure controlled, temp controlled extruder and the temp controlled table top.

FIG. 45 (c) The printer in the process of printing and the printed specimens.

Table Legends

Table 1. Extracellular osteocalcin secretion by ionic dissolution products of various metal oxide doped FP glass in MG 63 cells.

Table 2. Intracellular osteocalcin secretion by ionic dissolution products of various metal oxide doped FP glass in MG 63 cells.

Table 3. Preparation of scaffold by varying the proportion of FP glass.

Table 4. Effect of cell adherence over the scaffold (as in Table 4) in MG 63 cell lines.

Table 5. Preparation of scaffold by increasing the percentage of bioactive components PPF and FP GLASS at the cost of reducing the bioinert component PLA and PDLLA.

Table 6. Effect of cell adherence over the scaffold (as in Table 5) in MG 63 cell lines.

Table 7. Preparation of scaffold by varying the percentage of the Bioinert components PLA and PDLLA keeping the Bioactive components fixed (PPF and Glass powder).

Table 7a. Preparation of scaffold by varying the percentage of the Bioinert components PLA and PDLLA keeping the Bioactive components fixed (1,2-Diol and Glass powder).

Table 8. Effect of cell adherence over the scaffold (as in Table 7) in MG 63 cell lines.

Table 8a. Effect of cell adherence over the scaffold (as in Table 7a) in MG 63 cell lines.

Table 9. MTT of SaOS2 cell line varying according to the variation in the components and to the presence of pores.

Table 10a and 10b: Composites and the proportion of the components in the composite.

Table 11. MTT of the 12 types scaffolds (varying components, +/− PPF/XPPF, +/− Pores) in

SaOS 2 Cell line.

Table 12. ALP activity of composites.

Table 13. Chondroitin levels of scaffolds.

Table 14. Comparison chart of the biological activity of scaffolds.

Table 15. Invitro studies—pH variation of the simulated body fluid (SBF) of scaffolds over 21 days.

Table 16. XRD results of the compression moulded scaffold after SBF immersion.

Table 17. XRD results of the rapid heating scaffolds after SBF immersion.

Table 18. Percentage of crystallisation of scaffold in the in-vitro study.

### DETAILED DESCRIPTION

The invention is for a synthetic composite for a bone graft comprising of: bio inert polymers comprising poly lactic acid, poly D, L-Lactic acid; bio active polymer consisting of polypropylene fumarate or diester of fumaric acid and propylene diol (1,2-Diol); and a bioactive inorganic component consisting of a metal fluorophosphates glass powder wherein the amount of the bioactive components is upto 30% (w/w) of the composite.

The bioactive inorganic metal fluorophosphates glass powder of the composite is one of zinc fluorophosphate, magnesium fluorophosphate or silver fluorophosphate.

The polylactic acid in the composite is in the range of 54% (w/w) to 68% (w/w); poly D, L-lactic acid is in the range of 10% (w/w) to 28% (w/w); 1,2 diol is in the range of 3% (w/w) to 10% (w/w); polypropylene fumarate is in the range of 3% (w/w) to 10% (w/w); the inorganic metal fluorophosphates glass powder in the composite is in the range of 10% (w/w) to 30% (w/w).

In an aspect the composite comprises of polylactic acid, 1,2 diol, and zinc fluorophosphate.

In an aspect the composite comprises of polylactic acid, poly D, L-Lactic acid, 1,2 diol and zinc fluorophosphate.

In an aspect the composite comprises of polylactic acid, poly propylene fumarate and zinc fluorophosphate.

In an aspect the composite comprises of polylactic acid, poly D, L-Lactic acid, poly propylene fumarate and zinc fluorophosphate.

In an aspect the composite comprises of polylactic acid, 1,2 diol and magnesium fluorophosphate.

In an aspect the composite comprises of polylactic acid, poly D, L-Lactic acid, 1,2 diol and magnesium fluorophosphate.

In an aspect the composite comprises of polylactic acid, poly propoylene fumarate and magnesium fluorophosphate.

In an aspect the composite comprises of polylactic acid, poly D, L, lactic acid, poly propylene fumarate and magnesium fluorophosphate.

In an aspect the composite comprises of polylactic acid, 1, 2 diol and silver fluorophosphate.

In an aspect the composite comprises of polylactic acid, poly D, L-Lactic acid, 1,2 diol and silver fluorophosphate.

In an aspect the composite comprises of polylactic acid, poly propylene fumarate and silver fluorophosphates.

In an aspect the composite comprises of polylactic acid, poly D, L, lactic acid, poly propylene fumarate and silver fluorophosphate.

The composite is in the form of a powder, or a scaffold. The scaffold is a strip or a cylinder or a tube and the like as and when fabricated.

The synthetic composite of the invention is prepared by the following method comprising the steps of: mixing the composite in a solvent with the magnetic stirrer or sonicated to obtain a homogenous mixture; the mixture is casted over hot glass plate and the solution is brought to boil; and evaporation of the solvent by continuous boiling; and an interconnected porous scaffold with the homogenous distribution of the components of the composite was obtained.

In an aspect the solvent used in the method is one of dichloromethane, acetone, or toluene, or chloroform.

The porosity of the scaffold ranges from 20%-40%.

In addition, the scaffold is also made of desired shape and desired porosity by a custom-made 3D printer by the Direct Ink Printing Technique.

The method of obtaining a scaffold by a custom-made 3D printer by the Direct Ink Printing Technique comprising the following steps: The components of the composites are homogenised and cooled to 10-30° C. The chamber temperature is kept at 30-40° C. The ink is loaded into a pressure-controlled, non adherent extruder. The input writing details were fed to the printer through the microchip. The nozzle diameter was fixed as 300 μm for easy extrusion. The volumetric flow rate was set at 5 mm³/s. The space between the lines of writing was 200 microns and the number of layers are designed 10. The desired shape, thickness, porosity, layers fed by a computer is written on a hot plate (100° C.). The movement of the hot plate in x, y, z direction were also pre-set and the commands was transferred by the microchip.

In an aspect the biological evaluation of the fluorophosphate glass was ascertained by their MTT, their intracellular and extracellular osteocalcin secretion and also ALP secretion in relation to MG63 cell lines.

In an aspect the significance of the pores in the scaffold was assessed by calceinAM study and MTT evaluation.

In an embodiment the biological potential of the different composites with different composition of the components have been ascertained by the MTT of the composites in relation to the SaOS2 and Human Endothelial cell lines, their efficiency in enhancing secretion of Alkaline phosphatase, Chondroitin sulphate the ground substance in the bone.

In addition, the ability of the composites in the secretion of osteocalcin, collagen 11, RUN_X2 were assessed by RT-PCR method. The porosity in the multi-layered scaffold was assessed by MicroCT evaluation.

In an embodiment, invitro study of the various composites and the various scaffolds were done by immersing in SBF for 21 days and were then studied by their XRD, FTIR, SEM, and MicroCT.

In an embodiment the bone forming efficacy of the composite was assessed by in-vivo evaluation in rabbits, confirmed by histopathological evaluation.

In an aspect the synthetic composite has following characteristics

a) Biocompatible; b) Bioactive; c) Biodegradable; d) Nontoxic to the recipient; e) Bioconductive; f) Bioinductive; g) Bioconvertible; h) Rate of degradation to match the rate of bio-conversion; i) sterilisable; j) easy to be produced in bulk; k) workable to the desired shape; 1) cost effective

### EXAMPLES

The following examples are for the purpose of illustration of the invention and are not intended in any way to limit the scope of the invention.

Materials:

Poly lactic acid (PLA) and poly DL-lactic acid (PDLLA) were procured from BioDegmer® Japan. Polypropylene fumarate (PPF) and diester of fumaric acid and propylene diol (1,2 Diol) was procured from Department of Polymer Technology, Kamaraj College of Engineering and Technology, S.P.G.C. Nagar, K. Vellakulam-625 701, India.

The polymers have been synthesized at the Department of Polymer Technology, Kamaraj College of Engineering and Technology. The method involves addition of diethyl fumarate, 1,2 propane diol, zinc chloride (catalyst) and hydroquinone (crosslinking inhibitor) in reaction vessel in the molar ratio of 1.0:3.0:0.01:0.002. The reaction vessel was fitted with double walled condenser and the receiving flask connected to it for by product collection. The system was kept in an oil bath at 100° C. with efficient magnetic stirring with subsequent application of vacuum (−80 mmHg). The temperature was raised to 150° C. with constant stirring, esterification condensation reaction occurred. As a result of this, the intermediate bis (hydroxypropyl) fumarate diester was formed and ethanol was distilled as the primary by product. After the expected amount of ethanol collection, transesterification reaction was carried out with the elimination of excess amount of 1,2-propane diol as secondary by product. Now, the synthesized material was dissolved in acetone. This solution was repeatedly washed with ice cold distilled water to remove the unreacted reactants and catalyst. A sufficient amount of anhydrous sodium sulphate was added to the acetone solution of the polyester so as to dry the acetone solution. After filtration, the solvent was slowly evaporated in hot air oven at 50° C. to yield PPF.

Fumaric acid (1.0 mol) and 1,2-propane diol (2.2 mol) were taken in round bottom flask and p-toluene sulphonic acid was used as the esterification catalyst. The dry toluene was added to the reaction mixture in order to remove the water formed during the esterification as an azeotrope. The Dean Stark apparatus was used for the above purpose. The reaction vessel was submerged in an oil bath, mixed uniformly and continuously using magnetic stirrer. The temperature was initially set at 100° C. and gradually raised to 140° C. in 10° C. increments over a period of one hour. When the temperature reached 110° C. (boiling point of toluene), water starts to collect in the Dean Stark apparatus. The reaction was allowed to proceed till the stipulated quantity of water was collected. The material was purified by applying vacuum to remove the excess unreacted 1,2-propane diol and water.

The FP glass component of the invention was procured from Bone Substitutes, Madurai. The method of preparation is as outlined in Indian patent application 5760/CHE/2013, 5990/CHE/2013, 5989/CHE/2013 and cited as references for the preparation of FP glasses. The method is briefly outlined below. The measured quantities of the required chemicals (Na₂CO₃, CaCO₃, CaF₂, P₂O₅ and ZnO/Ag₂O/MgO) were taken in a ball mill and homogenised. The mixture was heated in the alumina crucible for 1 h upto 120° C. and cooled to room temperature. It was again ball milled for 1 hr. The components were taken in a platinum crucible and kept in a furnace preheated to 1100° C. and allowed for 90 mts. Then the crucible was sunk into a bowl having liquid nitrogen. The formed glass was broken to pieces and milled for 48 h to obtain nano powder of the specific fluorophosphates glass. The FP glass material was prepared at Bone Substitutes, Madurai, India.
The MG-63 (ATCC® CRL-1427™) were obtained from The National Centre for Cell Science (NCCS), Pune, INDIA
The Saos-2 (ATCC® HTB-85™) was obtained from The National Centre for Cell Science (NCCS), Pune, INDIA

### Example 1: Selection of Non Toxic Inorganic Metal Fluorophosphates Glass Powder

a) MTT Proliferation Assay: The MG-63 cells were cultured into 24 well plates and ionic dissolution products of metal doped bio glass (fluorophosphate (FP), Magnesium fluorophosphate (MgFP), Zinc fluorophosphate (ZnFP), Titanium fluorophosphate (TiFP), Zirconium fluorophosphate (ZrFP), Silver fluorophosphate (AgFP) and strontium fluorophosphate (SrFP)) were co treated with cells on 0 hr seeding and monitored till 48 h to study cell morphology and after that the cells were washed twice with 1×PBS before being incubated with 0.2 mg/mL of MTT (3-(4,5-dimethylthaizole-2-yl)-2,5-diphenyl tetrazolium bromide) for 2 h. The purple colored product formed was then dissolved with isopropyl alcohol and the optical density was measured at 570 nm using ELISA Reader (Robonik, India) (FIG.

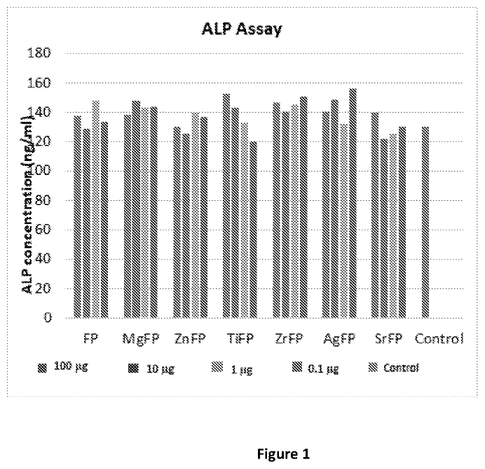

1). FIG. 1 shows all the metal oxide doped Fluorophosphate glasses were nontoxic and their viability exceeded 80% after 48 hrs of incubation (up to 10 microgram per ml.)

Alkaline phosphatase (ALP) is an essential enzyme in the process of bone formation from the mesenchymal cells to the mineralisation front. Hence its enhanced secretion is considered a vital factor to choose the ingredient for the composite for Bone Tissue Engineering (BTE). The results of the study in ALP secretion shows AgFP, ZrFP and MgFP showed consistently raised levels at all concentration from 0.1-100 μg/mL. ZnFP showed increased secretion only at lower concentrations of 0.01 and 1 μg/mL (FIG. 1).

b) Thermal Evaluation:

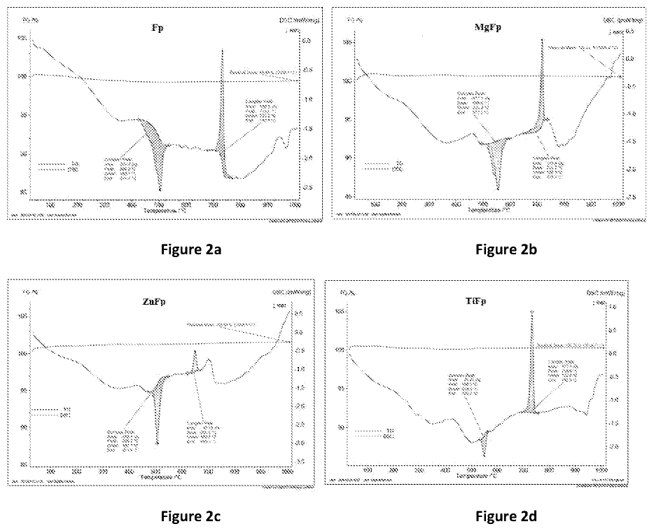

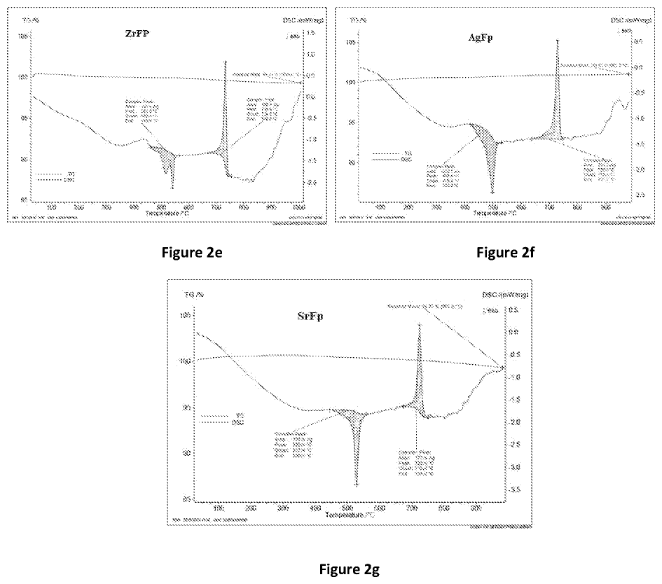

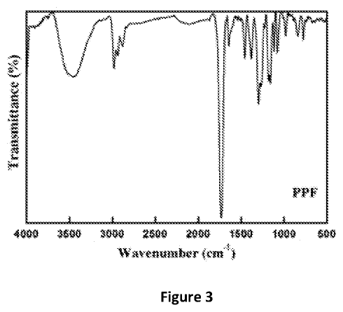

The Simultaneous Thermal Analysis (STA 449 F3Nevio) was used to obtain the thermal stability of the bioglass. 3.5 mg of metal fluorophosphates (each) was heated till 1000° C. at 50K min⁻¹ in nitrogen atmosphere. (FIG. 2a-2g).

The thermal evaluation of all the seven types of FP Glasses revealed that their Tg (glass transition temperature) was between 500-550° C. and their Tc (crystallisation temperature) was around 700° C. indicating a large window width of around 150° C. which can be useful in sintering while scaffolding (FIG. 2a-2g).

c) Osteocalcin (Intracellular and Extracellular) Assay by ELISA Method:

Osteocalcin secreted by MG-63 in response to the addition of ionic dissolution products of each fluorophosphates bio glass samples in 100 μg, 10 μs and 1 μg concentrations into the culture (extracellular as wells as intracellular) and responses were analysed by ELISA.

MG-63 cells were seeded into 24 well plates (2×10⁵ cells/well). After overnight adherence, media was removed and washed with Dulbecco's PBS. Ionic dissolution products of various Fluorophosphate bioglass samples with various concentrations were added to the wells (media without phenol red, serum and antibiotic). The assay plates were kept in CO₂ incubator with 5% CO₂ at 37° C. for 72 h. After incubation, supernatants were taken for the analysis of osteocalcin expression in extracellular environment.

For the assessment of intracellular expression of osteocalcin, cells from the wells were detached using Accutase (Gibco) and collected. 200 μL of cell lytic solution (Sigma) was added to each well and incubated for 10 m. Lysed cellular components were centrifuged and supernatant was taken for intracellular assessment. 100 μL from each sample was taken for evaluation by the ELISA method. Experiment was performed according to the instructions provided by the manufacturer (DIA source hOST-EASIA Kit, Belgium). Absorbencies were read at 450 nm. The expression of osteocalcin was calculated by plotting standard curve and values were expressed in ng/mL (Table 1 &2).

TABLE 1
Extracellular osteocalcin secretion by ionic dissolution
products of various metal oxide doped FP glass in MG 63 cells

| | | Extracellular (concentration in ng/mL) | | | |
|---|---|---|---|---|---|
| S. No | Sample | 100 μg | 10 μg | 1 μg | Control |
| 1 | FP | 0.179 | 0.492 | 0.247 | 0.430 |
| 2 | Mg FP | 0.317 | 0.300 | 0.469 | 0.430 |
| 3 | Zn FP | 0.324 | 0.557 | 0.208 | 0.430 |
| 4 | TiFP | 0.392 | 0.323 | 0.326 | 0.430 |
| 5 | ZrFP | 0.257 | 0.421 | 0.271 | 0.430 |
| 6 | Ag FP | 0.274 | 0.194 | 0.319 | 0.430 |
| 7 | SrFP | 0.306 | 0.302 | 0.220 | 0.430 |

Table 2 showing extracellular expression of osteocalcin in ng/mL

| | | Intracellular (concentration in ng/mL) | | | |
|---|---|---|---|---|---|
| S. No | Sample | 100 μg | 10 μg | 1 μg | Control |
| 1 | FP | 0.009 | 0.100 | 0.039 | 0.083 |
| 2 | Mg FP | 0.207 | 0.084 | 0.091 | 0.083 |
| 3 | Zn FP | 0.000 | 0.251 | 0.011 | 0.083 |
| 4 | TiFP | 0.083 | 0.000 | 0.288 | 0.083 |
| 5 | ZrFP | 0.003 | 0.099 | 0.005 | 0.083 |
| 6 | Ag FP | 0.253 | 0.028 | 0.096 | 0.083 |
| 7 | SrFP | 0.033 | 0.246 | 0.049 | 0.083 |

Bone is a composite of the ground substance reinforced by multiple collagens and mineralised by hydroxyl apatite. Though various collagens are present in various parts of the body osteocalcin is found exclusively in bone. It is also an excellent gene marker of bone induction. The ability of the ionic dissolution products of various FP glasses in various concentration were evaluated for their efficiency to promote osteocalcin secretion. While the extra cellular expression of osteocalcin showed increase than the control only with ZnFP and MgFP, (Table 1) intracellular osteocalcin was raised in most of the glasses but significant raise was present in ZnFP, MgFP and AgFP glasses and was more when the concentration of the products of dissolution was 10 μg/mL (Table2).

Based on the above studies and the characteristics of the fluorophosphate glasses AgFP, ZnFP, MgFP were selected for the composite preparation and for the fabrication of a composite into desired structure and shape.

### Example 2

a) Selection of Biopolymers and Characterization

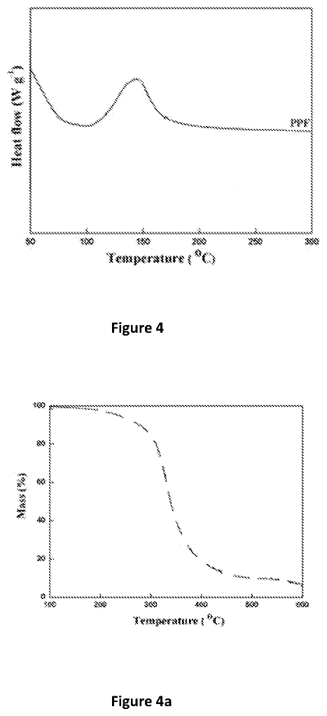

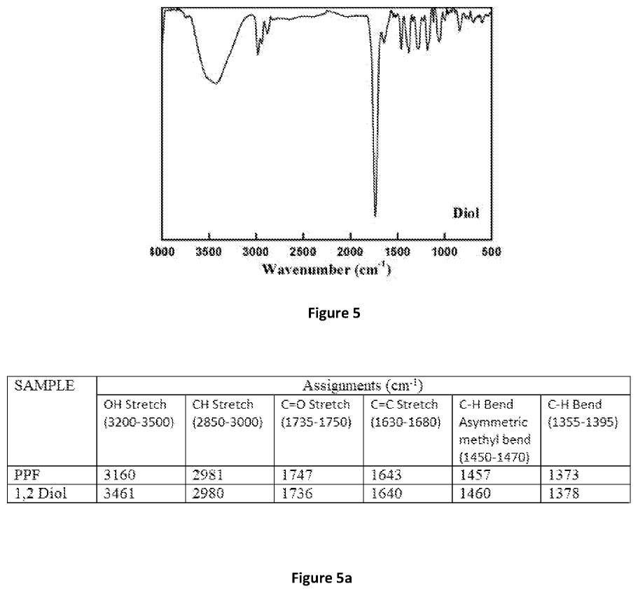

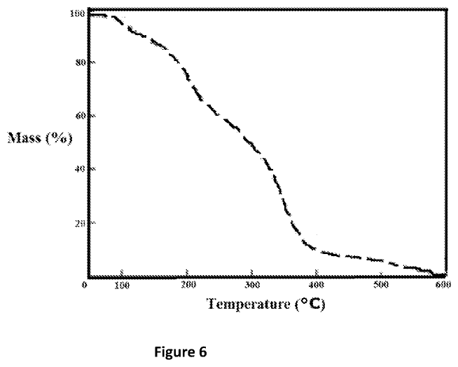

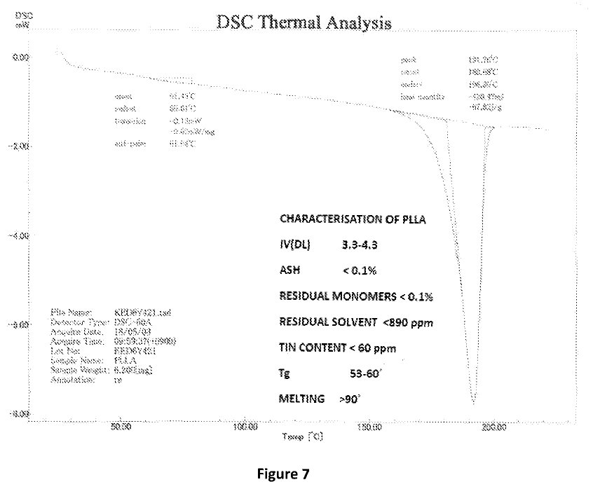

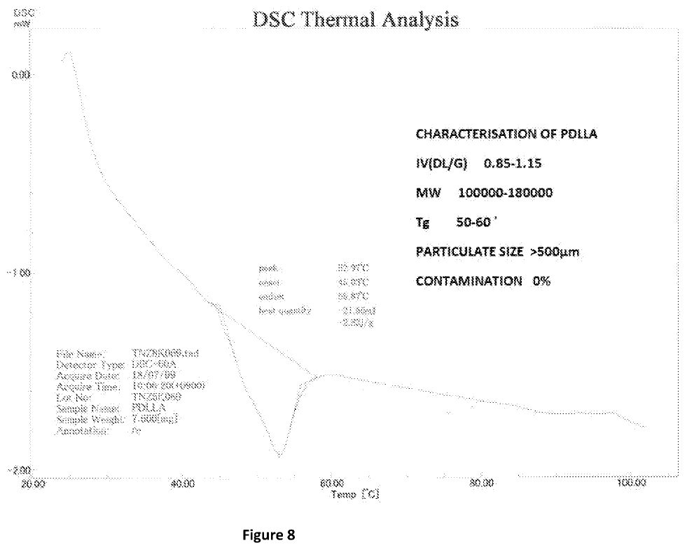

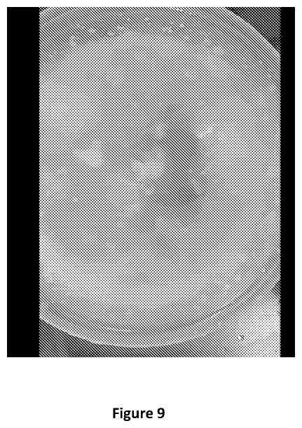

The bioinert and bioactive polymers were characterised for their properties. PPF and 1,2 Diol synthesized at the Department of Polymer Technology, Kamaraj College of Engineering and Technology, S.P.G.C. Nagar, K. Vellakulam-625 701, India. The medical grade PLA and PDLLA were procured from BioDegmer® Japan. The structural characterization (FTIR-84005 spectrophotometer, Shimadzu, Japan) and thermal evaluation (TA instruments DSC Q20) were carried out (FIG. 3-8).

b) Assessment of the Effective Percentage of FP Glass

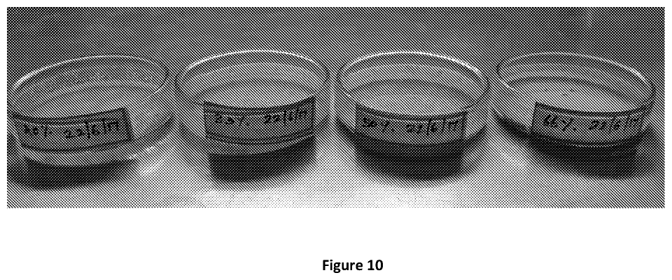

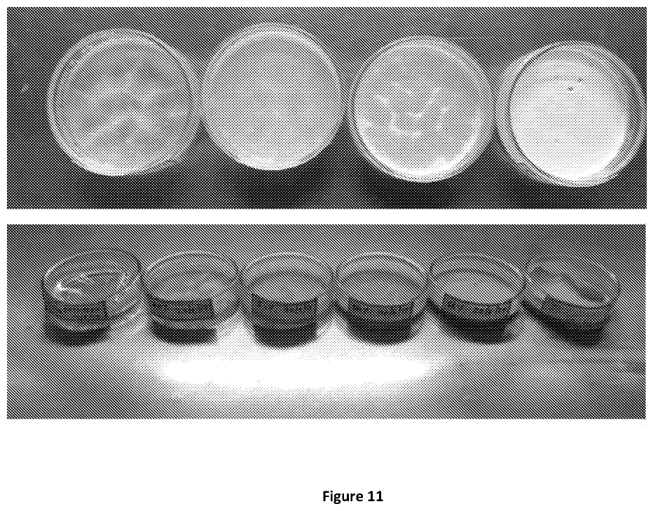

The optimum percentage of the FP glass was assessed by varying the proportions of the glass powder (0, 20, 33.3, 50, 66 and 75%) in the composite (FIGS. 10 & 11). The strength and ductility of the prepared material was examined manually. Also, the cell attachment was assessed in the composites as in the previous study to choose the right percentile of the glass powder (10, 12.5, 15, and 17.5%) (Table 3 & 4)

TABLE 3

| | | | | | | | GLASS | |
|---|---|---|---|---|---|---|---|---|
| | | | | | | | POWDER | |
| S.NO. | PLA g(%) | | PDLLA g(%) | | PPF g(%) | | g(%) | |
| 1. | 5.6 | (56) | 2.8 | (28) | 0.6 | (6) | 1 | (10) |
| 2. | 6.15 | (61.5) | 2.05 | (20.5) | 0.525 | (5.25) | 1.25 | (12.5) |
| 3. | 6.46 | (64.6) | 1.615 | (16.15) | 0.425 | (4.25) | 1.5 | (15) |
| 4. | 6.736 | (67.36) | 1.189 | (11.89) | 0.325 | (3.25) | 1.75 | (17.5) |

TABLE 4

| | Samples (No. of cells attached | | | | Samples (No. of cells attached | | | | | | | |
|---|---|---|---|---|---|---|---|---|---|---|---|---|
| | on plate) | | | | on Scaffold) | | | | Dead cells | | | |
| Days | 1 | 2 | 3 | 4 | 1 | 2 | 3 | 4 | 1 | 2 | 3 | 4 |
| 1 | 50 | 300 | 2000 | 48500 | 100 | 300 | 500 | 1000 | 49950 | 49700 | 48000 | 500 |
| 7 | 200 | 550 | 4500 | 69000 | 170 | 400 | 700 | 1500 | 49800 | 49450 | 45500 | 800 |
| 14 | 500 | 700 | 6000 | 80000 | 150 | 350 | 550 | 1200 | 49500 | 49300 | 44000 | 300 |
| 21 | 900 | 1000 | 11000 | 100000 | 100 | 300 | 500 | 1000 | 49100 | 49000 | 39000 | 350 |

c) Effect of Increasing the Composition of the Biopolymers

Of the four biopolymers PLA and PDLLA are bio inert and PPF and 1,2 Diol and FP GLASS are bioactive. The contribution of the bioactive ingredients were increased in minimal propositions at the cost of the bio inert PLA. The PLA share was reduced from 63.69% to 53.89% in graded decrements. It was substituted by increasing the FP GLASS and five types of scaffold were made. They were incubated with MG63 cell lines for 21 days following the previously mentioned protocol and the amount of adherent cells and the dead cells were tabulated. (Table 5 & 6)

TABLE 5

| | | | | | | | GLASS | |
|---|---|---|---|---|---|---|---|---|
| | | | | | | | POWDER | |
| S.NO. | PLA g(%) | | PDLLA g(%) | | PPF g(%) | | g(%) | |
| 1. | 7.005 | (63.69) | 1.189 | (10.81) | 0.33 | (3.00) | 2.475 | (22.5) |
| 2. | 6.736 | (61.24) | 1.189 | (10.81) | 0.325 | (2.95) | 2.75 | (25) |
| 3. | 6.466 | (58.78) | 1.189 | (10.81) | 0.319 | (2.90) | 3.025 | (27.5) |
| 4. | 6.197 | (56.34) | 1.189 | (10.81) | 0.314 | (2.85) | 3.30 | (30) |
| 5. | 5.928 | (53.89) | 1.189 | (10.81) | 0.308 | (2.8) | 3.575 | (32.5) |

TABLE 6

| | Samples (No. of cells attached | | | | | Samples (No. of cells | | | | | | | | | |
|---|---|---|---|---|---|---|---|---|---|---|---|---|---|---|---|
| | on plate) | | | | | attached on Scaffold) | | | | | Dead cells | | | | |
| Days | 1 | 2 | 3 | 4 | 5 | 1 | 2 | 3 | 4 | 5 | 1 | 2 | 3 | 4 | 5 |
| 1 | 22000 | 45000 | 39000 | 18000 | 23000 | 1750 | 3000 | 2000 | 1500 | 2250 | 1000 | 400 | 300 | 700 | 350 |
| 7 | 26000 | 77000 | 43000 | 35000 | 38000 | 2200 | 4500 | 3500 | 2250 | 3200 | 1500 | 700 | 500 | 400 | 400 |
| 14 | 32000 | 84400 | 52000 | 47000 | 53000 | 3000 | 7000 | 5000 | 2750 | 4500 | 1200 | 250 | 300 | 300 | 200 |
| 21 | 50000 | 92000 | 75000 | 65000 | 71000 | 3500 | 10000 | 6500 | 3600 | 6700 | 1000 | 200 | 250 | 500 | 350 |

It was observed that the composites having 75% of glass were ductile and broke on bending. The composites with lesser percentage of the glass were not breaking and were elastic (FIG. 10,11). When the polymer group had a small percentage (3%) of PPF/1,2 Diol the composite was not breaking and this expressed not only the bioactive nature of PPF/1,2 Diol but also the ability of it to alter the physical nature of the composite.

d) Effect of Varying the Percentage of the PLA and PDLLA with Fixed PPF/1,2 Diol and FP Glasses:

Scaffolds were made with keeping the proportion of the bioactive components PPF/1,2 Diol and FP glass as constant and the proportion of the bio inert components were varied, PLA being increased at the cost of reducing the percentage of PDLLA and the cell adhesion study and the no of viable and dead cells assessed as in the previous study and tabulated (Tables 7, 7a & 8, 8 a)

TABLE 7

| | | | | GLASS |
|---|---|---|---|---|
| S.NO. | PLA g(%) | PDLLA g(%) | PPF g(%) | POWDER g(%) |
| 1. | 6.406 (58.24) | 1.519 (13.81) | 0.325 (2.95) | 2.75 (25) |
| 2. | 6.516 (59.24) | 1.409 (12.81) | 0.325 (2.95) | 2.75 (25) |
| 3. | 6.626 (60.24) | 1.299 (11.81) | 0.325 (2.95) | 2.75 (25) |
| 4. | 6.736 (61.24) | 1.189 (10.81) | 0.325 (2.95) | 2.75 (25) |
| 5. | 6.846 (62.24) | 1.079 (9.81) | 0.325 (2.95) | 2.75 (25) |

TABLE 7a

| | Samples (No. of cells attached | | | | | Samples (No. of cells | | | | | | | | | |
|---|---|---|---|---|---|---|---|---|---|---|---|---|---|---|---|
| | on plate) | | | | | attached on Scaffold) | | | | | Dead cells | | | | |
| Days | 1 | 2 | 3 | 4 | 5 | 1 | 2 | 3 | 4 | 5 | 1 | 2 | 3 | 4 | 5 |
| 1 | 38000 | 39500 | 42000 | 44000 | 35000 | 1500 | 2250 | 3000 | 3500 | 1250 | 1500 | 1200 | 1000 | 400 | 2000 |
| 7 | 68500 | 70000 | 74000 | 77500 | 63000 | 1750 | 2500 | 3300 | 4000 | 1700 | 2250 | 2000 | 1500 | 700 | 1500 |
| 14 | 75000 | 77000 | 81000 | 84000 | 69000 | 3500 | 4700 | 6500 | 7250 | 3900 | 700 | 500 | 400 | 250 | 1000 |
| 21 | 82000 | 85000 | 87000 | 90000 | 83000 | 8900 | 9000 | 9200 | 10500 | 8000 | 800 | 550 | 500 | 200 | 12000 |

TABLE 8

| | | | | GLASS |
|---|---|---|---|---|
| S.NO. | PLA g(%) | PDLLA g(%) | 1,2-Diol g(%) | POWDER g(%) |
| 1. | 6.406 (58.24) | 1.519 (13.81) | 0.325 (2.95) | 2.75 (25) |
| 2. | 6.516 (59.24) | 1.409 (12.81) | 0.325 (2.95) | 2.75 (25) |
| 3. | 6.626 (60.24) | 1.299 (11.81) | 0.325 (2.95) | 2.75 (25) |
| 4. | 6.736 (61.24) | 1.189 (10.81) | 0.325 (2.95) | 2.75 (25) |
| 5. | 6.846 (62.24) | 1.079 (9.81) | 0.325 (2.95) | 2.75 (25) |

TABLE 8a

| | Samples (No. of cells attached | | | | | Samples (No. of cells | | | | | | | | | |
|---|---|---|---|---|---|---|---|---|---|---|---|---|---|---|---|
| | on plate) | | | | | attached on Scaffold) | | | | | Dead cells | | | | |
| Days | 1 | 2 | 3 | 4 | 5 | 1 | 2 | 3 | 4 | 5 | 1 | 2 | 3 | 4 | 5 |
| 1 | 38000 | 39500 | 42000 | 44000 | 35000 | 1500 | 2250 | 3000 | 3500 | 1250 | 1500 | 1200 | 1000 | 400 | 2000 |
| 7 | 68500 | 70000 | 74000 | 77500 | 63000 | 1750 | 2500 | 3300 | 4000 | 1700 | 2250 | 2000 | 1500 | 700 | 1500 |
| 14 | 75000 | 77000 | 81000 | 84000 | 69000 | 3500 | 4700 | 6500 | 7250 | 3900 | 700 | 500 | 400 | 250 | 1000 |
| 21 | 82000 | 85000 | 87000 | 90000 | 83000 | 8900 | 9000 | 9200 | 10500 | 8000 | 800 | 550 | 500 | 200 | 12000 |

The cell adhesion to the scaffold and to the glass plate beneath by 1) Preparation of scaffold by varying the proportion of FP glass 2) Preparation of scaffold by increasing the percentage of bioactive components PPF/1,2 diol and FP GLASS at the cost of reducing the bioinert components 3) Preparation of scaffold by varying the percentage of the Bioinert components PLA and PDLLA keeping the Bioactive components fixed are provided in Tables 3, 5, 7, 8.

The best results were achived when the bio active components (PPF/1,2 Diol+FP glass) were in the range of 10-30% and the relatively bio inert components (PLA+PDLLA) were in the range of 54-67% (Table 4,6,7a and 8a.)

e) Biological Effect of Varying the Components:

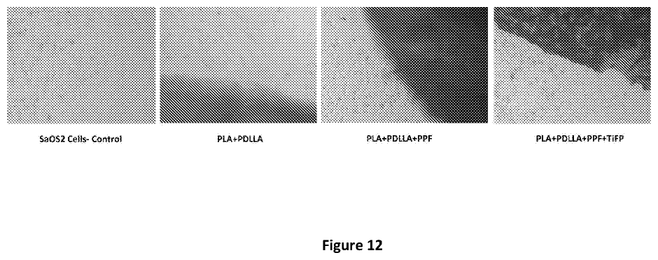

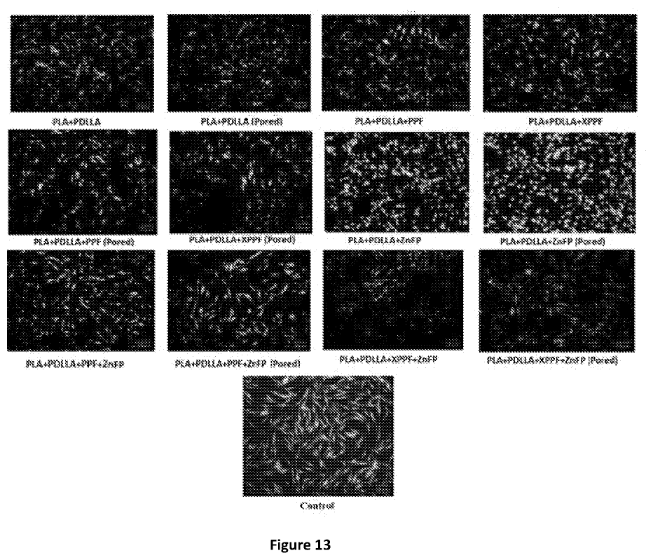

In order to assess how osteoblast-like cell line MG-63 adhere to scaffolds consisting of various combinations of polymers, Scaffolds of 1) PLA 2) PLA with PDLLA,3) PLA, PDLLA with PPF,4) PLA, PDLLA, PPF, with FP glass were made, cut into round shape of 12 mm using a cork borer, sterilized in UV, washed with PBS and immersed in serum-free MEM for an hour. Then scaffolds were transferred to 24 well plate and MG-63 cells (5×10⁴) were seeded onto the scaffolds. Plates were incubated in CO₂ incubator with 5% CO₂ at 37° C. for 1, 7, 14 and 21 days Minimum essential media (MEM) supplemented with 10% foetal bovine serum (FBS), 50 U/mL penicillin, 50 mg/mL streptomycin 1% L-glutamine (Gibco) was used throughout the experiment. To avoid the nutrient exhaustion, for the wells with 7, 14 and 21 days incubation, media was changed every two days. After incubation, the wells with scaffolds were observed and recorded as photographs using phase contrast microscope (Olympus, Japan) with the camera attached software (Magnus Analytics Mag Vision software; version—x 64, 3.7.6820) for maximum number of attached cells. (FIG. 12).

The evaluation conducted in MG63 cell lines underscores the bio activity of PPF and the FP glass. The cell adhesion which was nearly nil or negligible became obviously possible by the addition of a small quantity of PPF and was phenomenally increased by the addition of the FP glass. This effect is exhibited is shown in FIG. 12.

### Example 3: Porosity Assessment

a) Membrane Integrity (Calcein AM) Testing in Relation to Pored/Unpored, +/−PPF/XPPF, +/−FP Glass

Twelve different scaffolds were fabricated to assess the significance of the porosity. The membrane integrity and cell morphology of the cells was evaluated by double staining. 1*10⁵ SaOS2 cells were incubated with the testing material (12 mm diameter scaffold) for 24 h at 37° C. with 5% CO₂. The cell supernatant medium was aspirated out. The scaffolds gently washed with ice cold PBS solution and finally 2 μm Calcein AM added and incubated for 10 m at 37° C. The cells were examined under a microscope. (FIG. 13).

The calcein AM study to assess the cell wall integrity and the double staining to assess the cytotoxity showed interesting features. The control group of cells were not only brilliantly green but also showed homogenous spindle shape, indicating the integrity of cell wall and the metabolic potential. The addition of PPF to the basic components PLA+PDLLA increased the cell wall integrity and the addition of pores to the same increased the number of spindle shaped cells.

The addition of FP glass to the basic components PLA+PDLLA either pored are unpored increased the number of cells phenomenally but the quality of them were poor exhibited by their round shape rather than the spindle shape of the healthy cell.

When all the components PLA+PDLLA+PPF and FPglass were added both the intensity of fluorescence and the quality of the cells also increased and it was more so when pores were added to the composite.

The XPPF (auto polymerised PPF) when replaced the PPF in the composite there was only deleterious effect both in the fluorescence and the quality of the cells (FIG. 13)

b) MTT Assayin Relation to Pored/Unpored, +/−PPF/XPPF, +/−FP Glass

The MTT assay was used to evaluate mitochondrial activity of live cells. Cells were seeded in 12-well plates containing test materials at density of 1×10⁵ cells/well in 100 μL complete medium/well and incubated for 24 h at 37° C. After incubation, the cell culture media was aspirated, 10 μL MTT (5 mg/mL) was added to each well and incubated for 4 h. After wards, the resulting formazan crystals were solubilized in 100 μL/well of DMSO and quantified by measuring absorbance at 550 nm by Perkin Elmer microplate reader. Data were expressed as a percentage of control (untreated cells). (Table 9).

TABLE 9

| Sample name | Mean | SD | % cytotoxicity |
|---|---|---|---|
| PLA + PDLLA | 50.60 | 0.19 | 49.40 |
| PLA + PDLLA(Pored) | 48.21 | 1.21 | 51.79 |
| PLA + PDLLA + PPF | 43.70 | 0.78 | 56.30 |
| PLA + PDLLA + XPPF | 59.18 | 1.79 | 40.82 |
| PLA + PDLLA + PPF + ZnFP | 57.11 | 1.20 | 42.89 |
| PLA + PDLLA + XPPF + ZnFP | 42.70 | 1.36 | 57.30 |
| PLA + PDLLA + PPF(Pored) | 51.79 | 1.21 | 48.21 |
| PLA + PDLLA + XPPF(Pored) | 60.69 | 1.51 | 39.31 |
| PLA + PDLLA + PPF + ZnFP(Pored) | 78.37 | 3.92 | 21.63 |
| PLA + PDLLA + XPPF + ZnFP(Pored) | 69.53 | 1.71 | 30.47 |
| PLA + PDLLA + ZnFP | 54.23 | 0.47 | 45.77 |
| PLA + PDLLA + ZnFP(Pored) | 37.99 | 1.64 | 62.01 |

From the above study it can be inferred that the least toxic composite was that of PLA+PDLLA+PPF+FP glass (pored) (Table 9).

### Example 4: Composite Preparation and Scaffold Fabrication

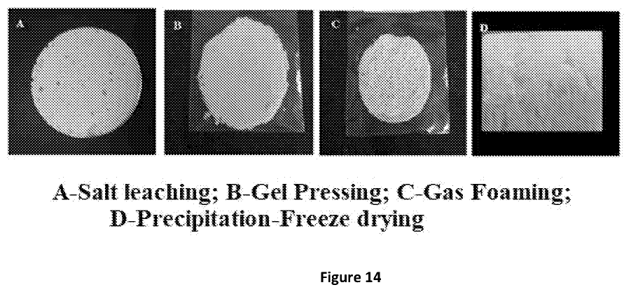

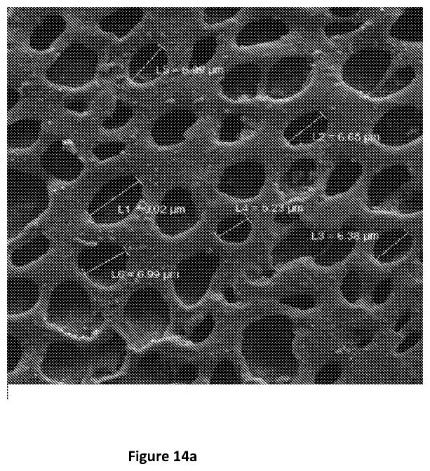

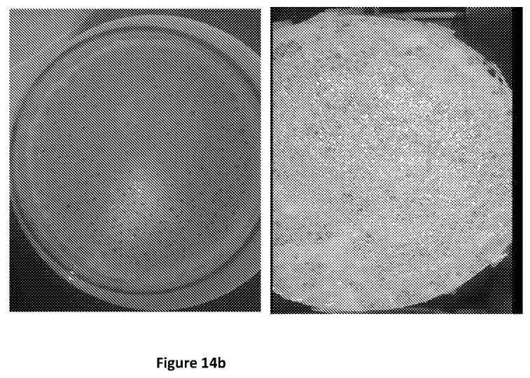

The four different methods were followed to prepare porous scaffold. (Salt leaching, Gas foaming, Gel pressing and Precipitation-Freeze Drying) (FIG. 14).

Salt Leaching:

The calculated amount of the PLA, PDLLA, PPF/Diol and AgFP/ZnFP/MgFP were taken and mixed with dichloromethane. The porogen (Sucrose-C₁₂H₂₂O₁₁) was sieved in the 300 and 100 μ mesh and it was added in 30% V/V basis. The porogen was mixed with the mixture using magnetic stirrer at 300 rpm. This slurry was poured into a teflon film coated petri dish and was placed in a warm chamber for 24 h. After drying, the film was compressed at 70° C. for 10 m. By sonication, the porogen was leached out using double distilled water. The prepared scaffold was dried in laminar air hood.

Gas Foaming

The calculated amount of the PLA, PDLLA, PPF/Diol and AgFP/ZnFP/MgFP were taken and mixed with dichloromethane. The porogen (Ammonium bicarbonate—NH₄)HCO₃) was sieved in the 300 and 100μ mesh and it was added in 30% V/V basis. The porogen was mixed with the mixture using magnetic stirrer at 300 rpm. This slurry was poured into a teflon film coated petri dish and was placed in a warm chamber for 24 h. After drying, the film was immersed in hot water, CO₂ emission occurred which inturn generates pores. Once all the bubbles settle down, the scaffold was placed in ice cold ethanol for 2 m. The fabricated scaffold was dried under laminar air hood for 24 h.

Gel Pressing

The calculated amount of the PLA, PDLLA, PPF/Diol and AgFP/ZnFP/MgFP were taken and mixed with dichloromethane. The porogen (Sucrose-C₁₂H₂₂O₁₁) was sieved in the 300 and 100μ mesh and it was added in 30% V/V basis. The porogen was mixed with the mixture using magnetic stirrer at 300 rpm. This slurry was poured into a teflon film coated petri dish and was placed in a warm chamber for 24 h. After complete evaporation of the solvent, the two films were pasted with methylene chloride and it was compressed at 70° C. for 10 minutes. By sonication, the porogen was leached out using double distilled water. The prepared scaffold was dried in laminar air hood.

Precipitation-Freeze Drying

The calculated amount of the PLA, PDLLA, PPF/Diol and AgFP/ZnFP/MgFP was taken and mixed with dichloromethane. The solution was slowly poured in to ice cold ethanol (non-solvent) under efficient stirring. The fibril like precipitate was obtained and it was washed with the double distilled water. The precipitate was packed into the cylindrical tube. The obtained precipitate was centrifuged at 3000 rpm for 15 m and it was kept in freezer for 12 h. The scaffold was freeze dried for 8 h.

Gel Foam Casting Under Rapid Heating

The required amount of the PLA, PDLLA, PPF/Diol and AgFP/ZnFP/MgFP were taken and mixed with dichloromethane under magnetic stirrer at 300 rpm. Once the mixture homogenised, the composite was slowly poured over a hot glass plate (70° C.). The solution started to boil and emitted the dichloromethane. With solvent evaporation, random pores were generated. The constant, continuous boiling kept the composite homogenous in spite of the difference in the densities of the four components. After complete evaporation, highly interconnected porous scaffold with homogenous distribution of the components was obtained. (FIG. 14a, 14b). The scaffold thus made can be marsealised to a powder, or cut into strips, or rolled into a cylinder. The same procedure were repeated with different solvents acetone, toluene, and chloroform and the same result was achieved.

The essential problem in homogenising the components was that all the three polymers chosen were soluble only in organc solvents and the essential bioactive inorganic component was highly hydrophilic and was soluble only in water. The other problem faced in homogenising the components was the gross difference in their densities. The other pre requiste apart from homogenesity was the essential need of pores and interconnecting pores for better bioactivity. The convenentional methods like salt leaching, gas leaching, gel pressing, precipitation and freeze drying all failed to achieve the desired homogenesity and the porosity. The highly dense FPglass powder settled in the base layer of the composite (FIG. 14). Also at the end of all these procedures the pores which had been designed to be around 200 microns got squeezed to around 10 microns because of the compression (FIG. 14a). But when Gel foam casting and rapid heating, two methods were combined the constant boiling gave the needed homogenesity and the vaporisation of the solvent gave the desired pores (FIG. 14b).

The following composites were prepared according to the Gel Foam Casting under Rapid Heating mentioned above and evaluated.

TABLE 10a

| Composite | | | | | | | |
|---|---|---|---|---|---|---|---|
| no | PLA | PDLLA | PPF | 1,2 DIOL | ZnFP | MgFP | AgFP |
| 1 | ✓ | x | x | ✓ | x | x | ✓ |
| 2 | ✓ | ✓ | x | ✓ | x | x | ✓ |
| 3 | ✓ | x | ✓ | x | x | x | ✓ |
| 4 | ✓ | ✓ | ✓ | x | x | x | ✓ |
| 5 | ✓ | x | x | ✓ | ✓ | x | x |
| 6 | ✓ | ✓ | x | ✓ | ✓ | x | x |
| 7 | ✓ | x | ✓ | x | ✓ | x | x |
| 8 | ✓ | ✓ | ✓ | x | ✓ | x | x |
| 9 | ✓ | x | x | ✓ | x | ✓ | x |
| 10 | ✓ | ✓ | x | ✓ | x | ✓ | x |
| 11 | ✓ | x | ✓ | x | x | ✓ | x |
| 12 | ✓ | ✓ | ✓ | x | x | ✓ | x |

TABLE 10b

| | | | | | AgFp/ |
|---|---|---|---|---|---|
| | | | | | ZnFp/ |
| | PLA | PDLLA | DIOL | PPF | MgFp |
| Scaffolds | (%) | (%) | (%) | (%) | (%) |
| PLA + Diol + AgFp | 64 | — | 8.5 | — | 27.5 |
| PLA + PDLLA + Diol + AgFp | 59.69 | 9.81 | 5.0 | — | 25.5 |
| PLA + PPF + AgFp | 64 | — | — | 8.5 | 27.5 |
| PLA + PDLLA + PPF + AgFp | 59.69 | 9.81 | — | 5.0 | 25.5 |
| PLA + Diol + ZnFp | 64 | — | 8.5 | — | 27.5 |
| PLA + PDLLA + Diol + ZnFp | 59.69 | 9.81 | 5.0 | — | 25.5 |
| PLA + PPF + ZnFp | 64 | — | — | 8.5 | 27.5 |
| PLA + PDLLA + PPF + ZnFp | 59.69 | 9.81 | — | 5.0 | 25.5 |
| PLA + Diol + MgFp | 64 | — | 8.5 | — | 27.5 |
| PLA + PDLLA + Diol + MgFp | 59.69 | 9.81 | 5.0 | — | 25.5 |
| PLA + PPF + MgFp | 64 | — | — | 8.5 | 27.5 |
| PLA + PDLLA + PPF + MgFp | 59.69 | 9.81 | — | 5.0 | 25.5 |

### Example 5: Cytotoxic Evaluation of the Fabricated Composites as Scaffolds

MTT Assay:

The non-toxic nature of the fabricated scaffolds were assessed by Saos-2 cell line (ATCC-85). 5×10⁶ SaOS2 cells at passage 25 were incubated in control medium supplemented with 10% fetal bovine serum 200 mM L-glutamine, 10 mM ascorbic acid, β-phosphate, 100 U/mL penicillin and 100 μg/mL streptomycin. The cells were incubated for 24-48 h for confluency. The confluent SaOS2 cells were washed twice using 1×PBS. A dimension of 2×2 cm² of scaffold were placed in six well plates and incubated for 48 h. The morphology of the cells was observed under an inverted microscope. The scaffolds were removed carefully and MTT was added and incubated for 4 h. The resulting formazan crystal was dissolved using DMS. The OD values were measured at 405 nm in a micro plate reader and the reading was tabulated. (Table 11).

TABLE 11

| Composites | % of live cells |
|---|---|
| PLA + Diol + AgFP | 90.95 |
| PLA + PDLLA + Diol + AgFP | 79.84 |
| PLA + PPF + AgFP | 81.48 |
| PLA + PDLLA + PPF + AgFP | 81.13 |
| PLA + Diol + ZnFP | 89.72 |
| PLA + PDLLA + Diol + ZnFP | 84.36 |
| PLA + PPF + ZnFP | 93.42 |
| PLA + PDLLA + PPF + ZnFP | 57.20 |
| PLA + Diol + MgFP | 90.54 |
| PLA + PDLLA + Diol + MgFP | 81.48 |
| PLA + PPF + MgFP | 53.09 |
| PLA + PDLLA + PPF + MgFP | 86.42 |
| Control | 100 |

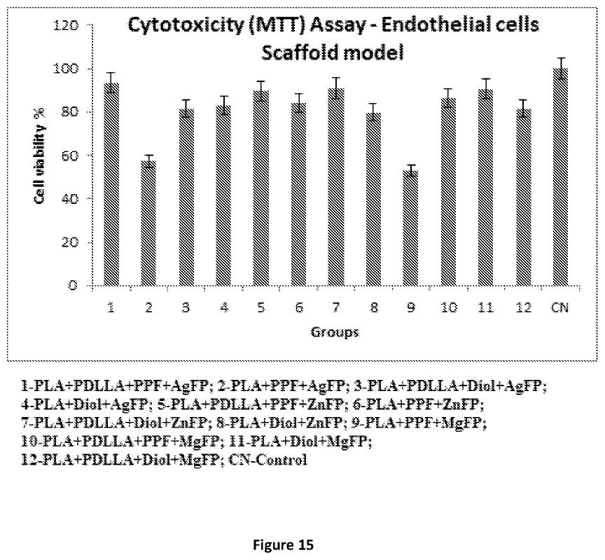

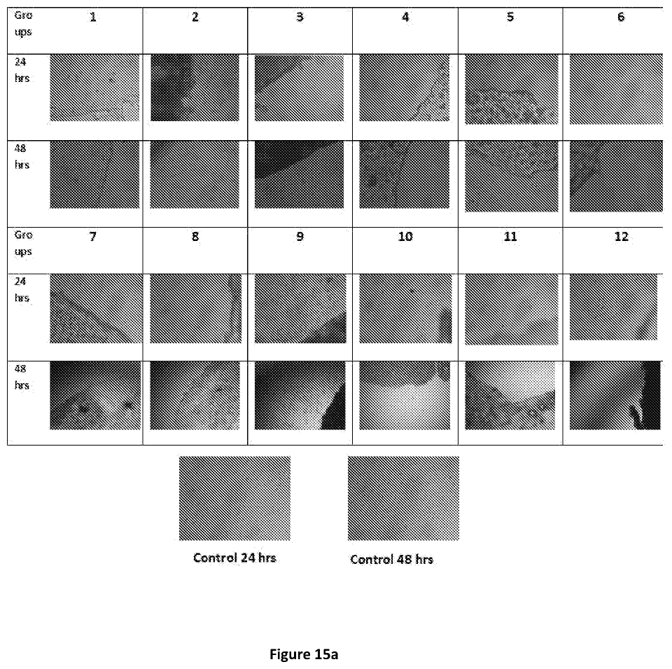

The same procedure was done with human endothelial cell lines and the results were noted with the morphological changes of the cells (FIGS. 15 and 15a).

The cytotoxicty of composites assessed by cytotoxic evaluation in both human endothelial cell lines and SaOS2 cell lines. With the HE cell lines except two composites (PLA+PPF+AgFP and PLA+PPF+ZnFP) all other composites showed viability more than 80% (FIG. 15, 15a). With SaOS2 cell lines except two composites (PLA+PDLLA+PPF+ZnFP and PLA+PPF+MgFP) all the other 10 had viability above 80%. (Table 11).

### Example 6: Biological Evaluation of the Fabricated Composites as Scaffolds

1) Alkaline Phosphatase (ALP) Activity of the Scaffolds

A 1*10⁶ MG63 cells were plated in culture plates and incubated for 48 h at 37° C. in 5% CO₂ incubator. Once the cells were confluent, it was treated with 2 cm*2 cm of each scaffold sample one in each well and incubated. After incubating for 48 h, cells were washed twice with ice cold PBS and homogenized in 504 assay buffer. The insoluble materials were centrifuged at 13,000 rpm for 3 min. The test samples with different concentrations of the exudates were added into 96-well plate and then 10 μL of ALP was added to each well. Then, 50 μL of the 5 mM pNPP solution was added to each well containing the test samples. The reaction mixture was incubated for 60 minute at 25° C. in dark condition. A 20 μL stop solution was added to terminate the ALP activity in the sample. The OD values are measured at 405 nm in a micro plate reader and the obtained results are noted in table. (Table 12).

TABLE 12

| Composites | ALP (IU/mL) |
|---|---|
| PLA + Diol + AgFP | 0.443 |
| PLA + PDLLA + Diol + AgFP | 1.915 |
| PLA + PPF + AgFP | 0.596 |
| PLA + PDLLA + PPF + AgFP | 1.406 |
| PLA + Diol + ZnFP | 0.938 |
| PLA + PDLLA + Diol + ZnFP | 2.306 |
| PLA + PPF + ZnFP | 0.503 |
| PLA + PDLLA + PPF + ZnFP | 1.543 |
| PLA + Diol + MgFP | 1.081 |
| PLA + PDLLA + Diol + MgFP | 1.208 |
| PLA + PPF + MgFP | 1.39 |
| PLA + PDLLA + PPF + MgFP | 1.544 |
| Control | 1.529 |

The ability of the fabricated composites to enhance the secretion of Alkaline phosphotase (ALP) in the SaOS2 cell lines was evaluated. ALP is a vital factor involved in most of the stages of Bone tissue formation. The values obtained showed that only two composites with 1,2, Diol and AgFP/ZnFP showed more secretion than the control while the composites with PPF were just at par with the control. The composites with MgFP, all the four types of them showed lower activity than that of the control (Table 12).

2) Evaluation of Osteocalcin, Collagen II, Run X2 Secretion of the Fabricated Composites as Scaffolds by RT PCR

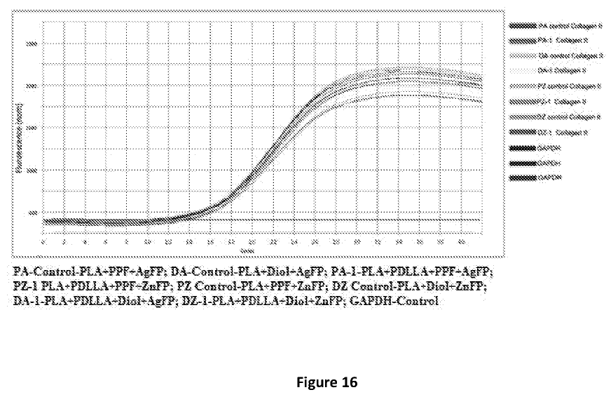

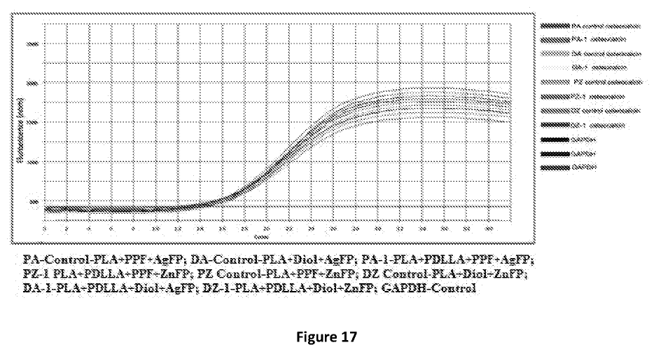

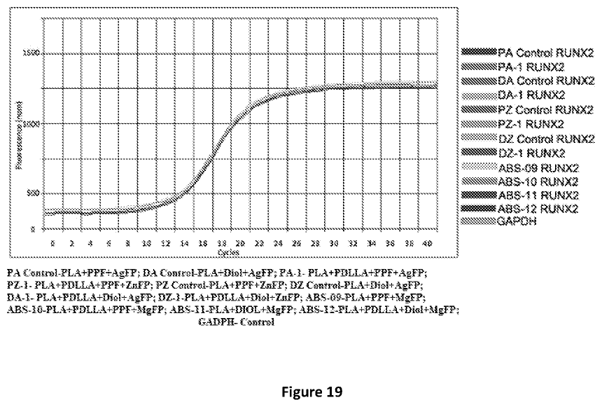

The study was done in SaOS2 cell lines. The total RNA was isolated from osteoblasts using TRIzol™ Reagent according to the manufacturer's protocol. The concentration of RNA was determined at 260/280 nm using NanoDrop spectrophotometer. For reverse transcription polymerase chain reaction (RT-PCR) the cDNA was synthesized by SuperScript™ First-Strand Synthesis System (Thermo Scientific) following the instructions provided. The synthesized cDNA was stored at 20° C. for later use. Simultaneous gene expression level for COL II (FIGS. 16 & 18), OCN (FIGS. 17 & 18), and Runx2 (FIG. 19) genes were measured by RT-PCR using SYBR green method.

Cycling Program of Temperature and Time

| | | | Number of |
|---|---|---|---|
| Phase | Duration (min) | Temperature (° C.) | cycles |
| Hold | 2 | 50 | 1 |
| Initial Denaturation | 10 | 95 | 1 |
| Denaturation, | 0.25, 0.5, 0.5 | 95, 60, 72 | 40 |
| Annealing | | | |
| Extension | 10 | 72 | 1 |

A real-time PCR reaction mixture (50 μL) preparation protocol followed as 25 μL SYBR Green Mix (2x), 0.5 μL liver cDNA, 2 μl primer pair mix (5 pmol/μl each primer) and 22.5 μL H₂O

The primers used for PCR were as follows:

Collagen Type II:

Forward primer:
CATGAGGGCGCGGTAGAGA
Reverse Primer:
ATCCCCTCTGGGTCCTTGTT

Product length: 296

Osteocalcein

Forward primer:
TCACACTCCTCGCCCTATTG
Reverse Primer:
CTCTTCACTACCTCGCTGCC

Product length: 132

Runx2Sequence (5′->3′) Template strand Length Start Stop Tm GC % Self complementarity

Self 3′ complementarity

Forward primer
CCACCGAGACCAACAGAGTC Plus
Reverse primer
GTCACTGTGCTGAAGAGGCT

Product length 119.

The analysis of the results was performed using ABI PRISM® 7000 Sequence DetectionSystem software that enables more sensitive and accurate estimation of the relative gene expression.

The results were tabulated. (Table 13).

TABLE 13

| | | | Collagen II | Osteocalcin | Run X2 | |
|---|---|---|---|---|---|---|
| | | ALP | (Fold | (Fold | (Fold | Chondroitin levels |
| Composites | MTT | (IU/mL) | increase) | increase) | increase) | (ng/mL) |
| PL A + Diol + AgFP | 90.95 | 0.443 | 0.16 | 2.3 | 0.04 | 7.45 |
| PLA + PDLLA + Diol + | 79.84 | 1.915 | 0.81 | 4.7 | 0.55 | 11.54 |
| AgFP | | | | | | |
| PLA + PPF + AgFP | 81.48 | 0.596 | 0.4 | 0.22 | 0.03 | 3.25 |
| PLA + PDLLA + PPF + | 81.13 | 1.406 | 4.4 | 3.2 | 0.5 | 5.43 |
| AgFP | | | | | | |
| PLA + Diol + ZnFP | 89.72 | 0.938 | 0.12 | 0.07 | 0.01 | 10.21 |
| PLA + PDLLA + Diol + | 84.36 | 2.306 | 1.7 | 1.2 | 0.4 | 3.85 |
| ZnFP | | | | | | |
| PLA + PPF + ZnFP | 93.42 | 0.503 | 0.5 | 1.4 | 0.09 | 13.23 |
| PLA + PDLLA + PPF + | 57.20 | 1.543 | 1.1 | 2.6 | 0.98 | 9.78 |
| ZnFP | | | | | | |
| PLA + Diol + MgFP | 90.54 | 1.081 | 0.25 | 0.05 | 0.78 | 10.88 |
| PLA + PDLLA + Diol + | 81.48 | 1.208 | 0.93 | 0.19 | 0.78 | 8.21 |
| MgFP | | | | | | |
| PLA + PPF + MgFP | 53.09 | 1.39 | 0.23 | 0.23 | 0.21 | 5.43 |
| PLA + PDLLA + PPF + | 86.42 | 1.544 | 0.53 | 0.24 | 0.2 | 10.21 |
| MgFP | | | | | | |
| Control | 100 | 1.529 | | | | 0.18 |

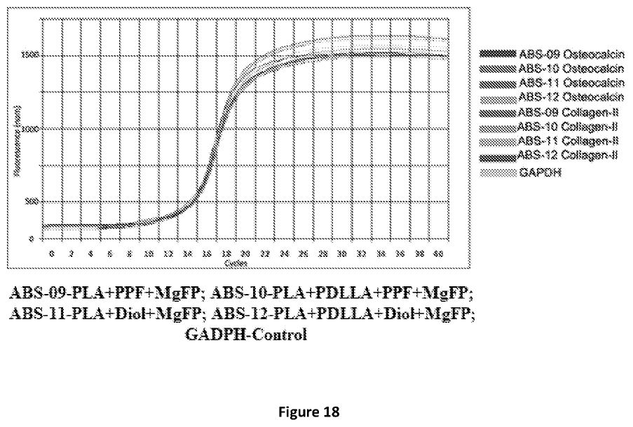

The three essential gene markers in the synthesis of bone from the stage of mesenchymal stemcells to that of the osteocyte maturation are OSTEOCALCIN, COLLAGEN II, and RUN-X2. When the results were charted to scrutinise the fold change than the control, the fold increase in collagen 11 was highest with PLA+PDLLA+PPF+AgFP and the highest fold increase in osteocalcin was also with AgFP but when constituted with 1,2, Diol than with PPF. The highest fold change in RUN_X2 than the control was with ZnFP when combined with PLA+PDLLA+PPF. All the Mg based composites fared poorly with all the three types of gene markers. (Table 13, FIGS. 16-19)

c) Chondroitin Sulphate Assay of the Scaffolds:

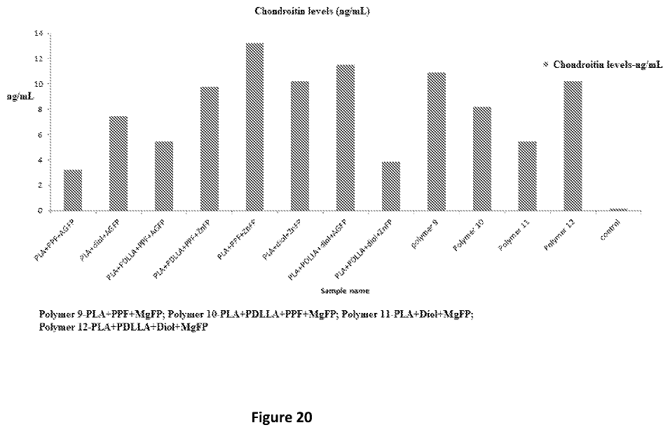

SaOS2 cell line was inoculated with the various composites for 48 hours. The cells were washed three times in cold PBS and suspended again in PBS (1x), frozen cells at ≤−20° C. and thawed. Repeated the freeze/thaw cycle 3 times.) Centrifuge at 1,500×g for 10 minutes at 2-8° C. to remove cellular debris. Chondroitin sulphate was measured using competitive ELISA method (Robonik, India). (FIG. 20) (Table 14).

TABLE 14

| Composites | Chondroitin levels (ng/mL) |
|---|---|
| PLA + Diol + AgFP | 7.45 |
| PLA + PDLLA + Diol + AgFP | 11.54 |
| PLA + PPF + AgFP | 3.25 |
| PLA + PDLLA + PPF + AgFP | 5.43 |
| PLA + Diol + ZnFP | 10.21 |
| PLA + PDLLA + Diol + ZnFP | 3.85 |
| PLA + PPF + ZnFP | 13.23 |
| PLA + PDLLA + PPF + ZnFP | 9.78 |
| PLA + Diol + MgFP | 10.88 |
| PLA + PDLLA + Diol + MgFP | 8.21 |
| PLA + PPF + MgFP | 5.43 |
| PLA + PDLLA + PPF + MgFP | 10.21 |
| Control | 0.18 |

The results showed all the twelve composites showed many fold increase in the secretion of CS than the control, immaterial of the component having 1,2, Diol or PPF and the FPglass being either Ag, Zn, or Mg. (Table 14).

### Example 7: In Vitro Evaluation of the Scaffolds

1) By following standard kokubo protocol, simulated body fluid (SBF) was prepared. All the fabricated scaffolds were cut into 2*2 cm² size. The scaffolds were placed in 20 mL SBF filled glass container, for a period of 21 days at 5% CO₂ incubator (Heraus—Germany). The pH variation was noted everyday using pH meter E1 model. After 21 days, the scaffolds were carefully removed; dried in laminar air flow for 48 h. The variation in the pH over 21 days of all the specimen were charted. (Table 15)

TABLE 15

| Samples | 1 | 2 | 3 | 4 | 5 | 6 | 7 | 8 | 9 | 10 | 11 |
|---|---|---|---|---|---|---|---|---|---|---|---|
| PLA_PPF_ZnFp | 6.63 | 6.64 | 7 | 7 | 6.91 | 6.97 | 6.93 | 7 | 7 | 7 | 7 |
| PLA_PDLLA_PPF_ZnFp | 6.5 | 6.7 | 7 | 7 | 7 | 6.93 | 6.93 | 7 | 7 | 7 | 7 |
| PLA_PPF_AgFp | 6.6 | 6.76 | 7 | 7 | 7 | 7 | 7 | 7 | 7 | 7 | 7 |
| PLA_PDLLA_PPF_AgFp | 6.7 | 6.82 | 7 | 7 | 7 | 7 | 7 | 7 | 7 | 7 | 7 |
| PLA_Diol_ZnFp | 6.57 | 6.79 | 7 | 7 | 7 | 7 | 7 | 7 | 7 | 7 | 7 |
| PLA_PDLLA_Diol_ZnFp | 6.7 | 6.74 | 7 | 7 | 7 | 6.92 | 6.93 | 7 | 7 | 7 | 7 |
| PLA_Diol_AgFp | 6.65 | 6.76 | 7 | 7 | 7 | 7 | 7 | 7 | 7 | 7 | 7 |
| PLA_PDLLA_Diol_AgFp | 6.71 | 6.78 | 7 | 7 | 7 | 7 | 7 | 7 | 7 | 7 | 7 |
| PLA_PPF_MgFp | 6.54 | 6.67 | 7 | 7 | 6.89 | 6.93 | 6.88 | 7 | 7 | 6.9 | 7 |
| PLA_PDLLA_PPF_MgFp | 6.53 | 6.76 | 7 | 7 | 6.85 | 6.9 | 6.89 | 6.93 | 6.91 | 6.92 | 6.92 |
| PLA_Diol_MgFp | 6.65 | 6.75 | 7 | 7 | 6.97 | 6.97 | 6.97 | 7 | 7 | 7 | 7 |
| PLA_PDLLA_Diol_MgFp | 6.6 | 6.72 | 7 | 6.93 | 6.84 | 6.82 | 6.81 | 6.89 | 6.88 | 6.87 | 6.89 |
| PLA_PDLLA_PPF_ZnFp(RH) | 6.74 | 6.82 | 7 | 7 | 6.93 | 7 | 7 | 7 | 7 | 7 | 7 |
| PLA_PDLLA_PPF_AgFp(RH) | 6.77 | 6.84 | 7 | 7 | 6.93 | 6.93 | 6.92 | 7 | 7 | 7 | 7 |
| PLA_PDLLA_PPF_MgFp(RH) | 6.76 | 6.85 | 7 | 7 | 6.91 | 6.91 | 6.9 | 7 | 6.92 | 6.93 | 7 |
| Single Layer | 6.9 | 7 | 7 | 7 | 6.92 | 7 | 7 | 7 | 7.1 | 7.09 | 7.09 |
| Multi Layer | 6.79 | 6.89 | 6.9 | 7 | 7 | 6.84 | 6.88 | 6.84 | 7 | 7 | 7 |

| Samples | 12 | 13 | 14 | 15 | 16 | 17 | 18 | 19 | 20 | 21 |
|---|---|---|---|---|---|---|---|---|---|---|
| PLA_PPF_ZnFp | 7 | 6.85 | 6.84 | 6.77 | 7 | 6.87 | 7 | 6.83 | 6.75 | 6.77 |
| PLA_PDLLA_PPF_ZnFp | 7 | 6.88 | 6.72 | 6.78 | 7 | 7 | 6.92 | 6.75 | 6.74 | 6.72 |
| PLA_PPF_AgFp | 7 | 6.87 | 6.75 | 6.88 | 7 | 7 | 6.82 | 6.78 | 6.75 | 6.78 |
| PLA_PDLLA_PPF_AgFp | 7 | 6.9 | 6.75 | 6.8 | 7 | 7 | 6.84 | 6.75 | 6.75 | 6.8 |
| PLA_Diol_ZnFp | 7 | 6.82 | 6.71 | 6.82 | 7 | 7 | 6.82 | 6.75 | 6.75 | 6.78 |
| PLA_PDLLA_Diol_ZnFp | 7 | 6.87 | 6.71 | 6.81 | 7 | 7 | 6.82 | 6.75 | 6.72 | 6.73 |
| PLA_Diol_AgFp | 7 | 6.93 | 6.77 | 6.78 | 7 | 6.92 | 6.92 | 6.76 | 6.75 | 6.76 |
| PLA_PDLLA_Diol_AgFp | 7 | 6.89 | 6.73 | 6.8 | 7 | 7 | 6.86 | 6.77 | 6.78 | 6.78 |
| PLA_PPF_MgFp | 6.92 | 6.79 | 6.64 | 6.68 | 6.89 | 6.81 | 6.74 | 6.69 | 6.68 | 6.66 |
| PLA_PDLLA_PPF_MgFp | 6.92 | 6.8 | 6.64 | 6.69 | 6.89 | 6.83 | 6.74 | 6.68 | 6.69 | 6.7 |
| PLA_Diol_MgFp | 7 | 6.83 | 6.71 | 6.75 | 7 | 7 | 6.85 | 6.74 | 6.77 | 6.76 |
| PLA_PDLLA_Diol_MgFp | 6.88 | 6.71 | 6.62 | 6.69 | 6.89 | 6.83 | 6.72 | 6.66 | 6.67 | 6.65 |
| PLA_PDLLA_PPF_ZnFp(RH) | 7 | 6.79 | 6.65 | 6.74 | 7 | 6.93 | 6.81 | 6.71 | 6.73 | 6.71 |
| PLA_PDLLA_PPF_AgFp(RH) | 7 | 6.79 | 6.68 | 6.74 | 7 | 6.93 | 6.83 | 6.72 | 6.74 | 6.69 |
| PLA_PDLLA_PPF_MgFp(RH) | 6.93 | 6.78 | 6.68 | 6.61 | 6.89 | 6.84 | 6.76 | 6.66 | 6.66 | 6.65 |
| Single Layer | 7 | 7.15 | 7 | 6.93 | 7 | 7 | 7.07 | 7 | 7 | 7 |
| Multi Layer | 7 | 7 | 6.83 | 6.84 | 6.8 | 6.85 | 7 | 6.86 | 6.83 | 6.86 |

The pH variation of all the compression moulded specimens showed uniformly a reduction in the first 2 days which is because of phosphoric acid formation. And all the specimens bounced back to 7 on the third day due to the alkaline earth metal (Na⁺ and/or Ca²⁺) release. The dissolution of the ions thus replaces H⁺ ions by cations (Na⁺ and/or Ca²⁺) leading to an increase in hydroxyl ion concentration. None of them went below 6.5 even in the first two days. From then on it showed a steady variation between 7 and 6.7.

The scaffolds, single layered strip and the multi layered cylinders made by Gel Foam Casting under rapid heating showed a better pHeven in the first two days and never went below 6.8 and the end stage also showed higher pHthan the compression moulded scaffolds. The highest pH reached was with the strip of scaffold made by rapid heating method and it was 7.15. This variation shows the better homogenesity and the porosity achived by the rapid heating method which avoids high acidic environment that can lead on to rejection (Table 15).

2) XRD

The pre and post immersion specimen (pre and post immersion refers to the scaffolds before immersion in the SBF and after immersion in SBF and drying) was subjected to XRD evaluation. The X-Ray Diffraction was captured using PANalyticalX′PertPRO powder X-ray Diffractometer The deposited materials crystal size was calculated semi-quantitatively by adopting Scherrer equation.

D=kλ/β cos θ

where:

D is the mean size of the ordered (crystalline) domains, which may be smaller or equal to the grain size, which may be smaller or equal to the particle size; (nm)

k is a dimensionless shape factor, with a value close to unity. The shape factor has a typical value of about 0.9, but varies with the actual shape of the crystallite;

λ is the X-ray wavelength; λ=0.15406 nm

β is the line broadening at half the maximum intensity (FWHM), after subtracting the instrumental line broadening, in radians.

θ is the Bragg angle. (Table 16)

TABLE 16

| | Pre immersion | Post immersion |
|---|---|---|
| Samples | (crystal size nm) | (crystal size nm) |
| PLA_PPF_ZnFP | 16.46 | 23.32 |
| PLA_PDLLA_PPF_ZnFP | 17.45 | 21.86 |
| PLA_PPF_AgFP | 14.07 | 22.60 |
| PLA_PDLLA_PPF_AgFP | 34.54 | 15.92 |
| PLA_Diol_ZnFP | 16.35 | 21.90 |
| PLA_PDLLA_Diol_ZnFP | 29.45 | 18.33 |
| PLA_Diol_AgFP | 16.11 | 28.62 |
| PLA_PDLLA_Diol_AgFP | 19.27 | 22.64 |
| PLA_PPF_MgFP | 21.44 | 20.74 |
| PLA_PDLLA_PPF_MgFP | 13.22 | 16.33 |
| PLA_Diol_MgFP | 25.86 | 20.71 |
| PLA_PDLLA_Diol_MgFP | 11.57 | 14.65 |

The XRD of the three specimens prepared by combining rapid heating and gel foam casting was evaluated for the pre immersion and the post immersion status. (Table 17).

TABLE 17

| | Pre immersion | Post immersion |
|---|---|---|
| Samples | (crystal size nm) | (crystal size nm) |
| PLA_PDLLA_PPF_ZnFP(RH) | 22.49 | 20.49 |
| PLA_PDLLA_PPF_AgFP(RH) | 30.81 | 13.94 |
| PLA_PDLLA_PPF_MgFP(RH) | 19.87 | 14.24 |
| STRIP (RH) | 26.47 | 50.29 |
| CYLINDER (RH) | 33.01 | 39.66 |

In the XRD evaluation, the crystal size (D) of the deposited material was calculated in both the pre immersion and the post immersion status by Schrrer equation. Among the pre immersion values, the crystal size of the PLA+PDLLA+PPF+AgFP showed the highest value. This is arributed to the high hydrophilicity of the composite and the reaction it has undergone with the atmospheric humidity during the waiting period of scaffolding and the evaluation. (Table 16) The same effect is observed in the scaffolds made by Gel foam casting under rapid heating method also and the highest size of the crystals were seen with the same composite of AgFP (Table 17).

3) FITR Analysis:

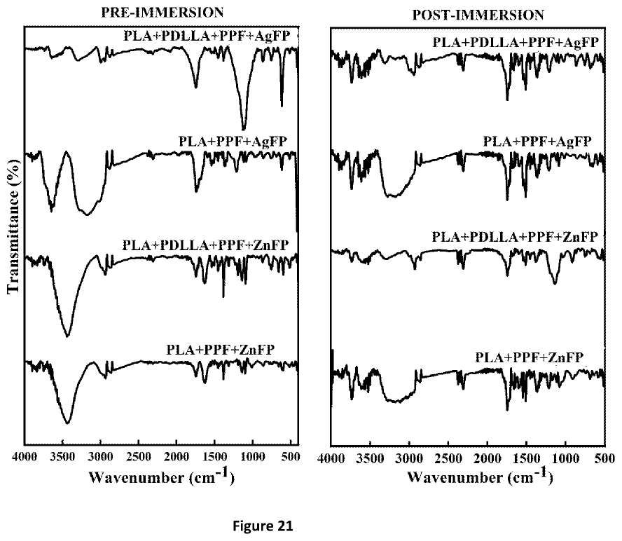

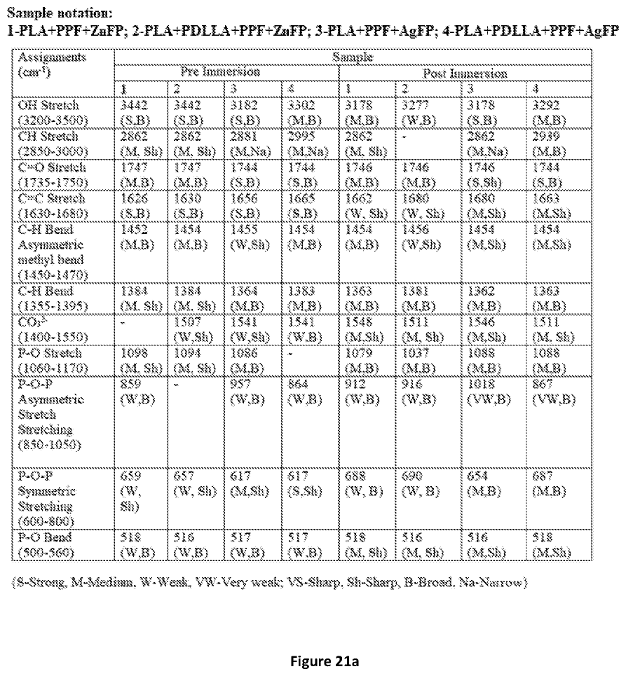

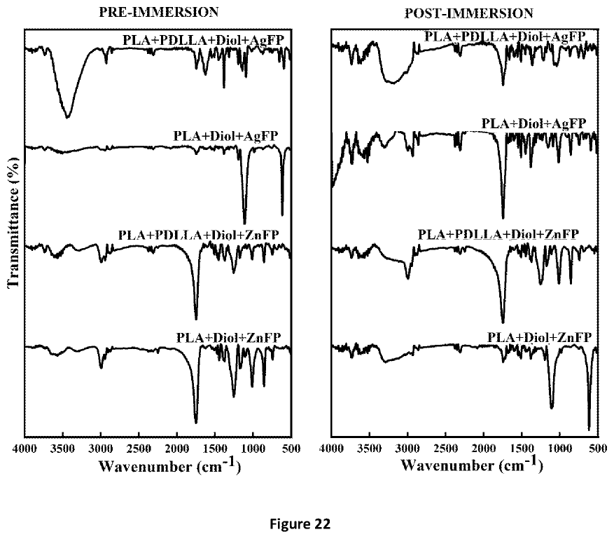

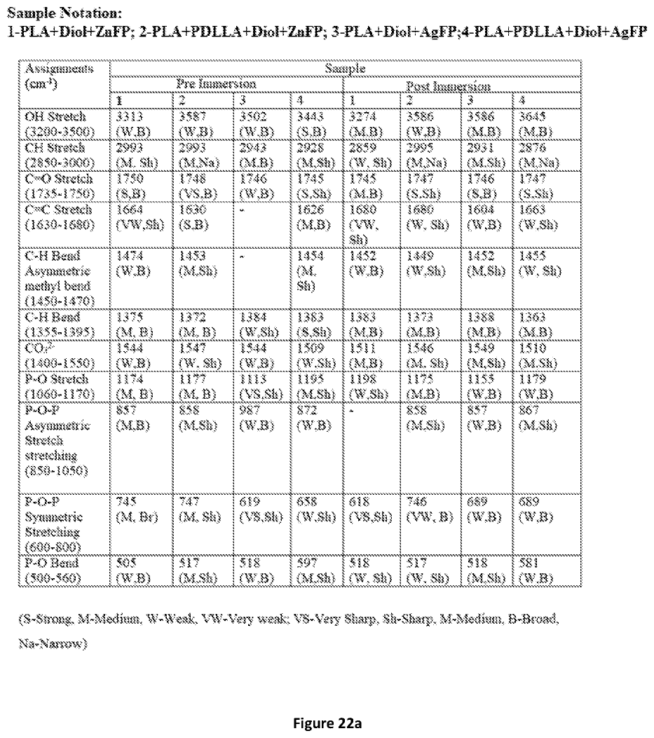

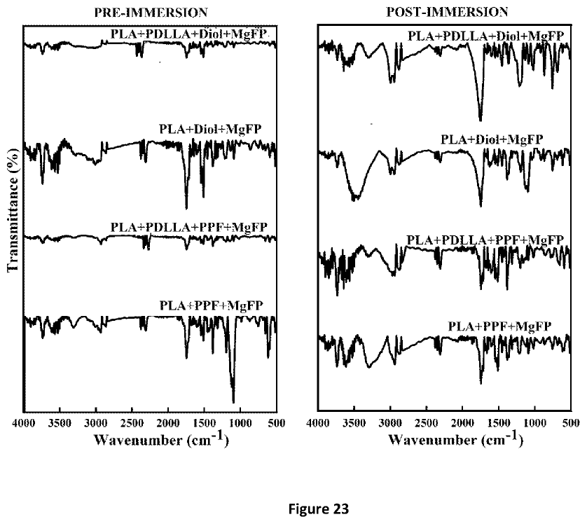

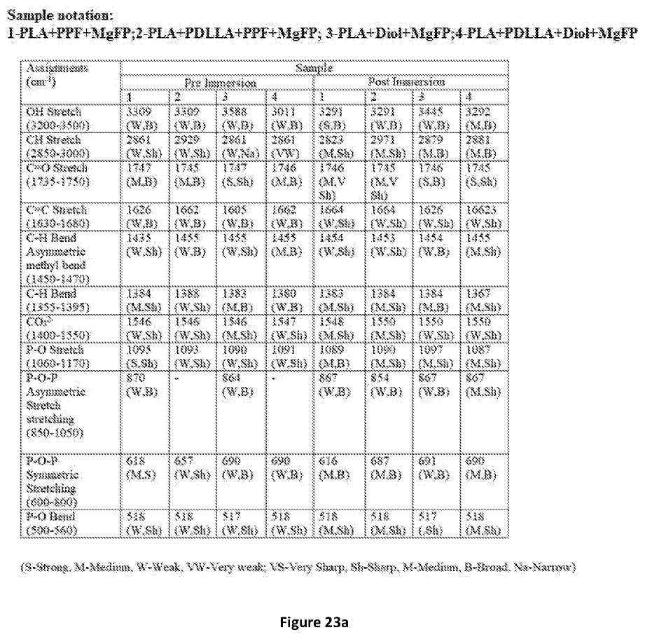

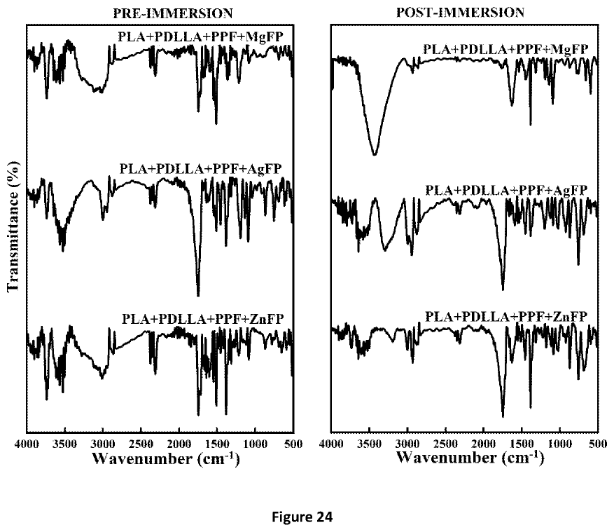

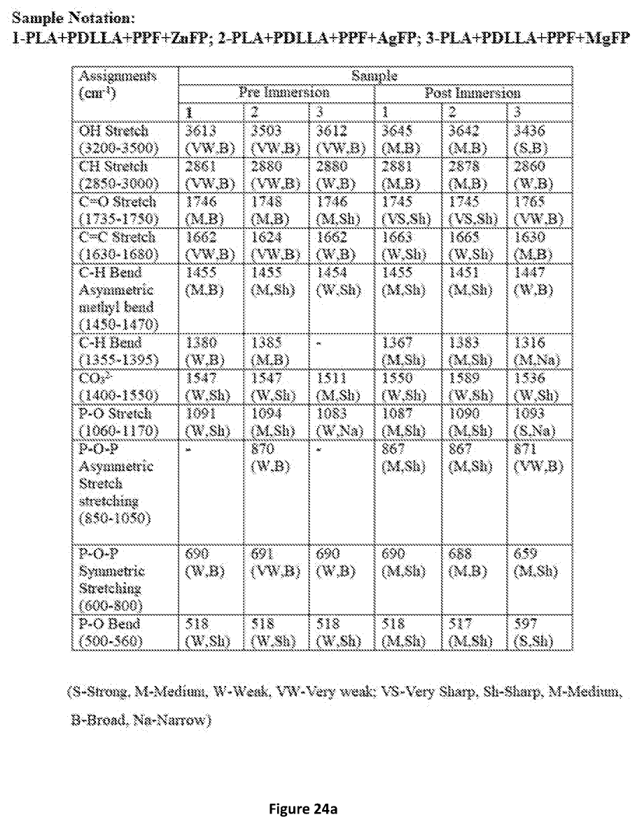

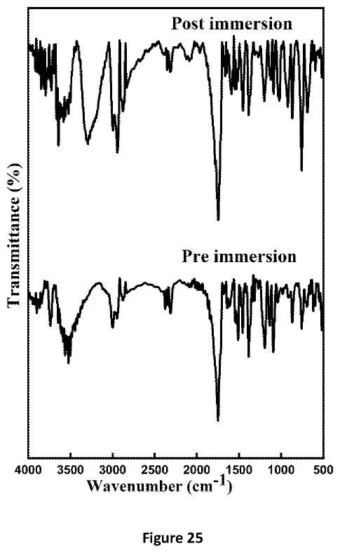

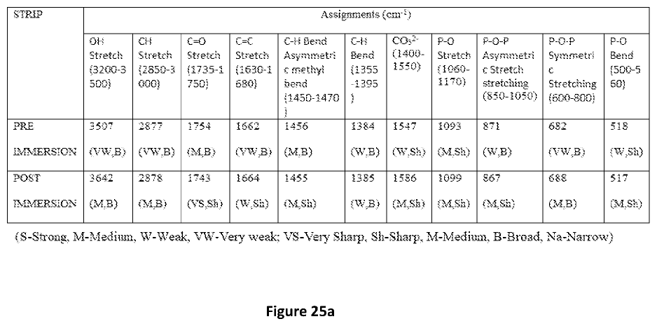

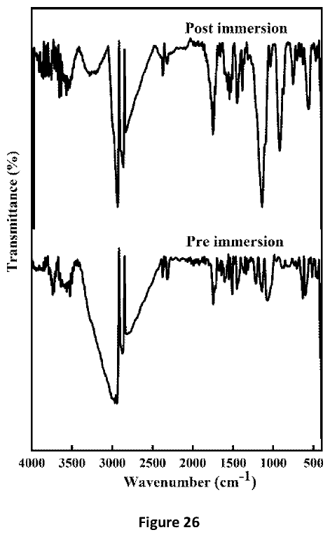

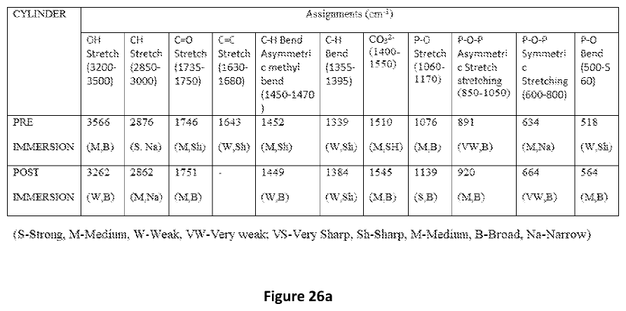

A Small amount of pre and post immersion specimen (pre and post immersion refers to the scaffolds before immersion in the SBF and after immersion in SBF and drying). were ground separately with potassium bromide and made into pellets. This pellets were used for the analysis. The analysis was done in the spectral range of 500-4000 cm⁻¹ by Fourier transform infrared-84005 spectrophotometer, Shimadzu, Japan. (FIG. 21-26).

The predominant functional groups of the composite seen in the FTIR evaluation by their respective spectral ranges are alcohol (3200-3500 cm⁻¹), alkanes (2850-3000 cm⁻¹), saturated ketone (1735-1750 cm⁻¹), alkenes (1630-1680 cm⁻¹), asymmetric methyl bend (1450-1470 cm¹) methyl bending (1350-1395 cm⁻¹¹). The presence of P—O bend (560-500 cm⁻¹) bands indicates the formation of calcium phosphate (CaO—P₂O₅) layer. The carbonate group (CO₃)²⁻(1400-1550 cm⁻¹) bands show the crystalline nature of the HA layer. The bands are observed at above 3500 cm⁻¹ which corresponds to the OH group. After 21 days of soaking in SBF the strong intensity and frequency shift of the (CO₃)²—, P—O—P stretch and P—O bend groups reveal the interaction of the composite and HAp precipitation. (FIG. 21-26, 21a-26a).

The shoulder peak at 1450-1410 cm⁻¹ coupled with the weaker peak at 870-875 cm⁻¹ corresponds to type B carbonate vibrations, whereas the vibration regions of type A carbonate are 1450-1410 cm⁻¹ coupled with a band at 880 cm⁻¹. The type A and B carbonate are indistinguishable in these scaffolds because of the ester peaks also lies on the same region. Both type A and B carbonates are present in these scaffolds and their intensities are maximum at three selected compression moulded scaffold composites (PLA+PDLLA+PPF+ZnFP, PLA+PDLLA+PPF+AgFP, PLA+PDLLA+PPF+MgFP.) For the same composites the corresponding peaks for HAp in rapid heating combined gel foam casting is higher than the compression moulded scaffolds.

Although the HAp precipitation was noted in all the fabricated scaffolds, the intensity of the carbonated group (CO₃)²⁻and phosphate based group (P—O—P asymmetric and symmetric stretch, P—O bend) was observed as very high in Gel foam casting under Rapid heating than those of the corresponding composites made by compression moulding. (FIG. 21a-26a)

4) SEM EDAX

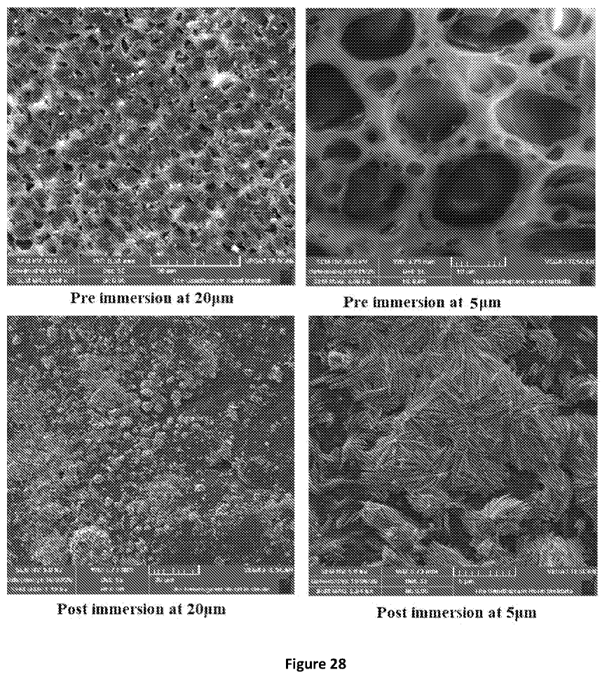

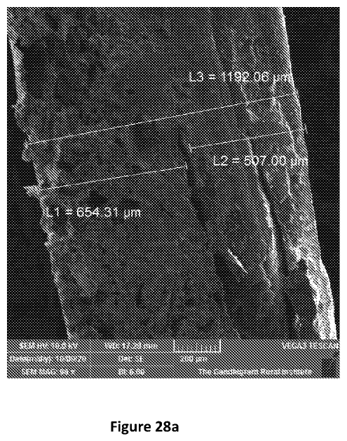

The morphological analysis and the semi-quantitative elemental concentration of the pre- and post-immersion samples prepared by compression moulding and pored by sugar leaching were examined using Scanning Electron Microscope—SEM (Model Ultra 55; Zeiss, Oberkochen, Germany) coupled with Energy Dispersive X-ray Spectrograph (ModelOxford Xmax50 EDS, Oxford Instrument, England) after gold sputtering. (FIG. 28)

The specimens were cut into two halves to expose the interior of the scaffold. The exposed interior surface was sputtered with gold and analysed using the same Scanning Electron Microscope. (FIG. 28a)

The width of crystallisation in relation to the actual width of the specimen was calculated as a percentage and has been tabulated to access the scaffold for Bio conversion. (Table 18).

TABLE 18

| Sample code | % of crystallinity |
|---|---|
| PLA + PPF + ZnFP | 87.31 |
| PLA + PDLLA + PPF + ZnFP | 76.19 |
| PLA + PPF + AgFP | 49.36 |
| PLA + PDLLA + PPF + AgFP | 72.7 |
| PLA + Diol + ZnFP | 59.54 |
| PLA + PDLLA + Diol + ZnFP | 78.89 |
| PLA + Diol + AgFP | 86.88 |
| PLA + PDLLA + Diol + AgFP | 97.42 |
| PLA + PPF + MgFP | 94.62 |
| PLA + PDLLA + PPF + MgFP | 82.09 |
| PLA + Diol + MgFP | 73.33 |
| PLA + PDLLA + Diol + MgFP | 78.68 |
| PLA + PDLLA + PPF + ZnFP(RH) | 73.62 |
| PLA + PDLLA + PPF + AgFP(RH) | 81.42 |
| PLA + PDLLA + PPF + MgFP(RH) | 87.34 |

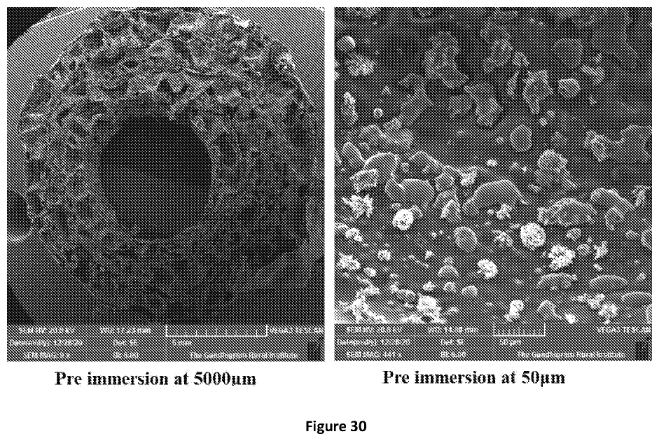

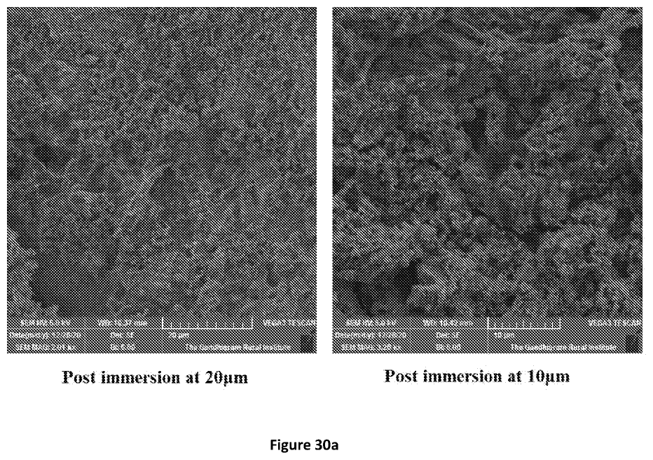

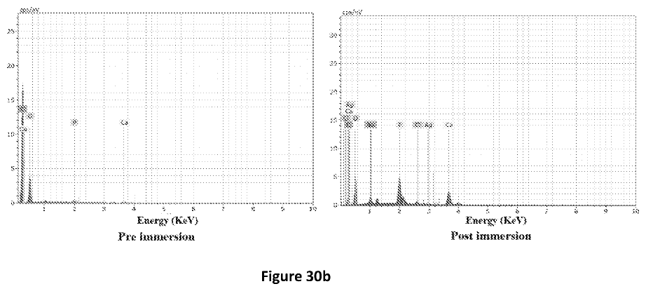

A single layer of the composite was made by Gel Foam Casting under Rapid Heating. A cylinder with a inner core diameter of 5 mm was made with the composite. SEM evaluation of the single layer specimen made by Gel Foaming under Rapid heating and the multi layered cylinder were done after gold sputtering. (Model Ultra 55; Zeiss, Oberkochen, Germany) (FIG. 30)

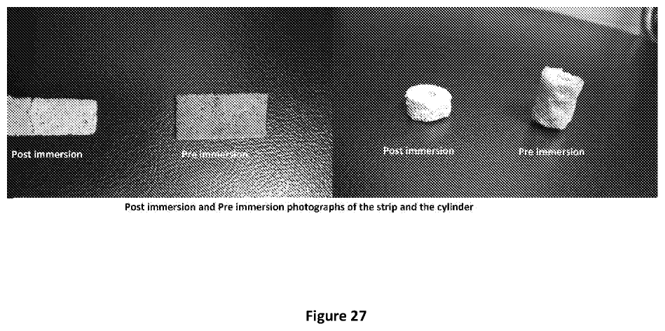

The similar specimens subjected to in vitro evaluation were analysed by the same way in the same Scanning Electron Microscope to assess the degree of surface pores and the change in crystallinity after in-vitro study (FIG. 30a). The clinical photograph of a stirp of composite and a cylindrical composite, both made by gelfoam casting under rapid heating shows the retention of the shape after SBF immersion for 21 days, but the complete change in the colour and the texture indicating the crystalline conversion (FIG. 27). The SEM of a compression moulded scaffold in two different magnification both before and after in vitro evaluation are shown in FIG. 28 which shows very scarce amount of crystallisation in the pre invitro evaluation and the homogenous pores being well exhibited. After 21 days of immersion in SBF the crystalline conversion is well seen and all the pores have been near completely clogged with the crystals formed.

The specimens after SEM study were broken into two halves and the interior was evaluated by SEM for the crystallisation percentage of the thickness (FIG. 28a). There was no significant change in the percentage and it can be inferred all the composites has near equal conversion once the pores allow penetration of the SBF inside except the absence of PDLLA had some significance in the extent of crystalline conversion (Table 18).

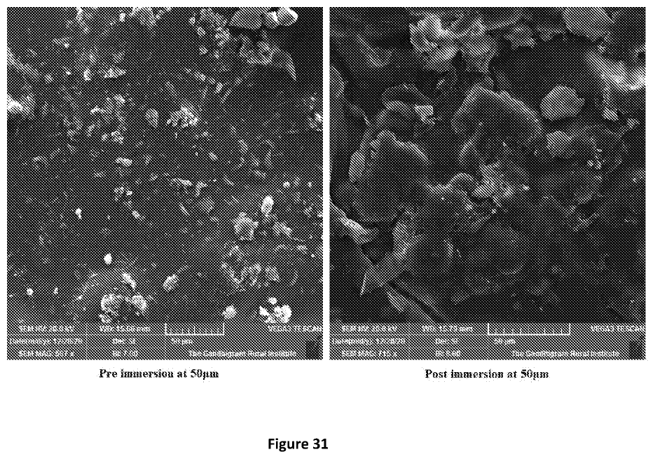

SEM study of a strip of scaffold made by gel foam casting under rapid heating in the pre in vitro status shows specks of crystallisation indicating the high hydrophilicity of the scaffold (FIG. 31) and the post immersion evaluation of the same shows complete conversion into crystallinity which proves the high bioresorbability of the scaffold (FIG. 31a)

The SEM evaluation of a cylindrical scaffold made by rapid heating under low magnification shows the adequacy of pores. This proves the homogenising of the polymers by the simple method adapted (FIG. 30). The pre in-vitro and the post in-vitro SEM clearly shows the complete crystallisation that has occurred. (FIG. 30a) The EDX evaluation of the pre and post in vitro SEM confirms the high level of carbonated hydroxy apatite formation in the scaffold (FIG. 30b).

5) MICRO—CT Evaluation

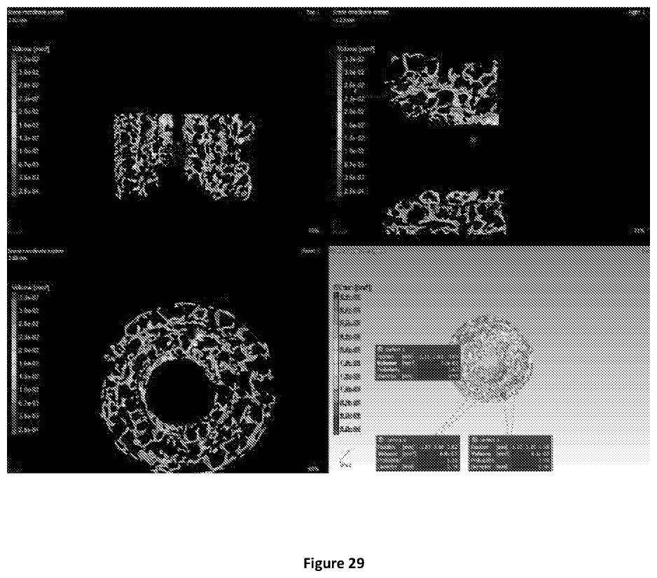

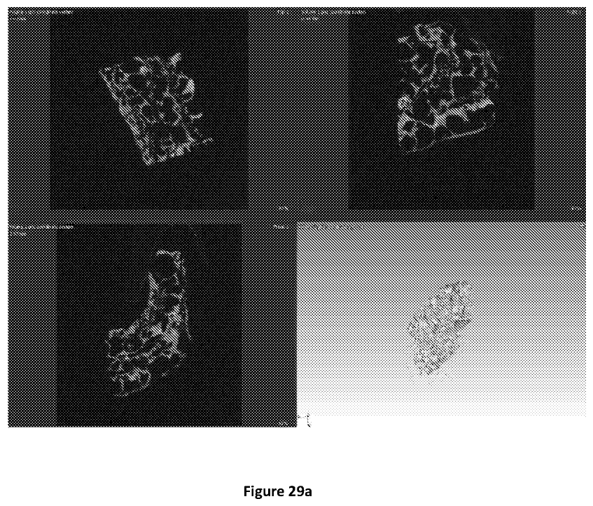

The surface and internal architecture of the scaffolds made in the single layer of strip, and the multi-layered cylinder were evaluated by the GE SRμCT analyser at various voxels and were 3D reconstructed. The porosity was assessed in all three planes (the axial, coronal and the sagittal). This disclosed the degree of porosity and the extent of the interporous connection. (FIG. 29).

The specimens were subjected to in-vitro evaluation (immersed in SBF under 5% CO₂ environment at 37° C. for 21 days) and the change was recorded by photograph (FIG. 27) The post immersion Micro-CT evaluation showed complete crystallisation. (FIG. 29a).

MICRO-CT evaluation of the cylindrical scaffold made by gel foam casting under rapid heating proved the following factors a) The scaffold had no layering and was continuous. b) There was adequate porosity and the pore sizes were varying. c) The pores were all well connected by interpores (FIG. 29). The same specimen after in vitro evaluations had complete conversion to crystalline nature with the preservation of the deeper pores (FIG. 29a).

### Example 8: In Vivo Evaluation of the Composite Granules

The in vivo studies were conducted with the Ethics committee approval (Ethical committee approval no ABS/IAEC/18 Oct. 2019/003-)

A single species of Orictologus cuniculus was purchased from King Institute, Chennai, India and domesticated over a period of two weeks. The day night rhythm was maintained and was fed on good nourishing food. The adaptation was confirmed by the gain in weight of 150 g in two-week time. (1800-1950 g). The composite (PLA+PDLLA+PPF+AGFP) granules were prepared by morcellation of the scaffold and sterilised by ethylene oxide gas.

The animal was given a premedication of pedichloryl syrup (2.5 ml) thirty minutes before surgery. Intra muscular ketamine anaesthesia was given in the dose of 45 mgs per kilogram body weight and waited for ten minutes to get the full dissociated anaesthetic effect. The anaesthetic effect was maintained by oxygen and sevoprim inhalation through mask

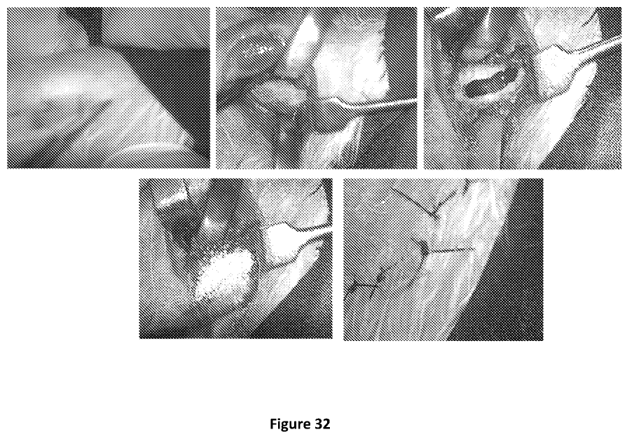

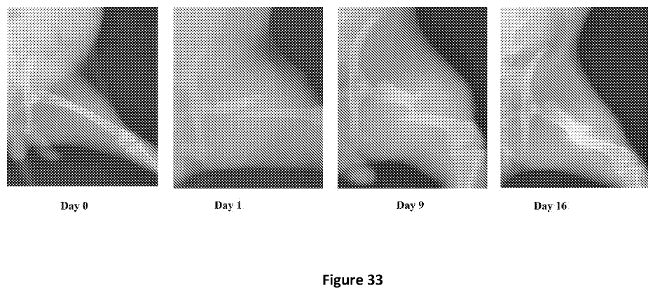

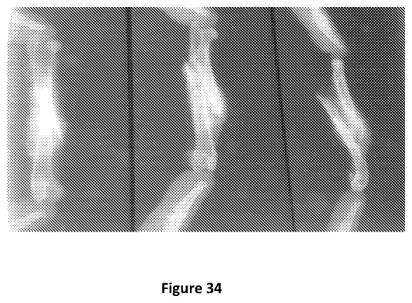

The left thigh was repeatedly painted with 10% povidone iodine and ethylene alcohol. Xylocaine 2% with adrenaline was injected in the line of incision as an additional analgesia and also a haemostatic agent. The skin incision made on the antero lateral aspect was rolled down to expose the posterior boarder of the quadriceps muscle. Using sharp dissection, the muscle was slit open and enlarged by thin bone spikes to expose the antero lateral aspect of the thigh bone. Using an electric dental burr of 1 mm a trough was made for a length of 2 cms. This exposed the medullary cavity. It was packed with the sterile composite powder. Liberal saline wash was given to wash off the spilled over composite materials. The spikes once removed the muscle fell back into position completely covering the bony trough. Two 3-0 vicryl stitches were used to close the muscle. The skin incision which was far away from the bone work was closed with 3-0 ethilon. A single dose of ceftrioxazone 250 mg was given intra muscularly. (FIG. 32). It was found that the femur had fractured and the ends were apart (FIG. 34). Neither immobilisation of the femur or any form of fixation was done. The rabbit was not limping and was feeding well. There was only a flare of the ends of the fracture and there was no evidence of any callus on the 9^{th} post-operative day. After another week (Day 16) the limb when examined clinically and it had a sound union. X-ray taken showed abundant callus not only in the fracture end but all along the femur where the trough had been made and even below (FIG. 33.)

The animal was euthanized, the limb harvested, skin and muscles were peeled off and an abundant amount of callus was found to have united the fracture very strongly. The dissected specimen was x-rayed and the specimen preserved in 10% formalin. (FIG. 34)

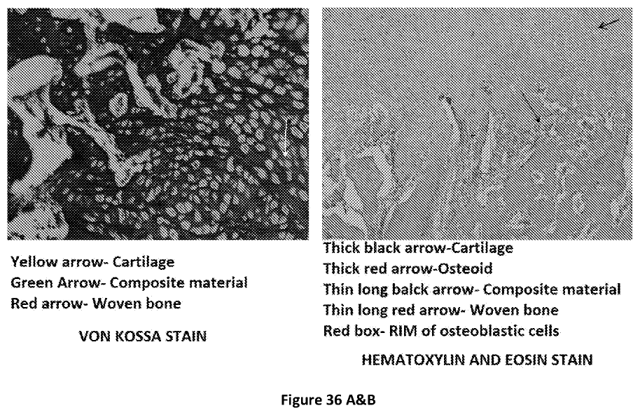

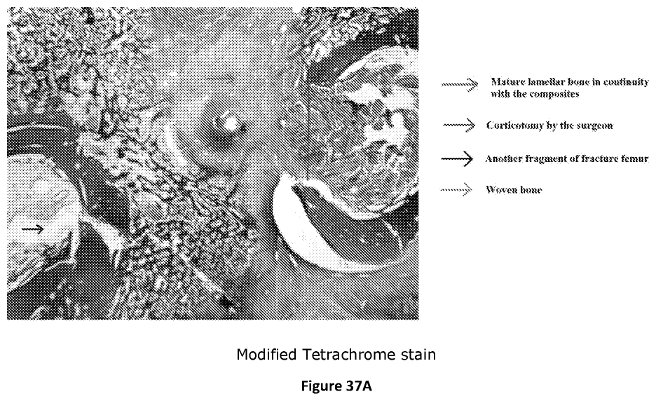

The specimen was prepared and the decalcified specimen was sectioned axially to exhibit the two segments of the femur with the intervening tissue formed. The specimen was stained using regular eosin-haematoxylin stain and also von kossa stain. (FIG. 36).

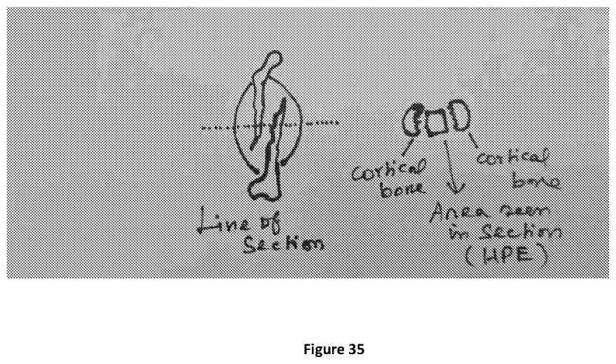

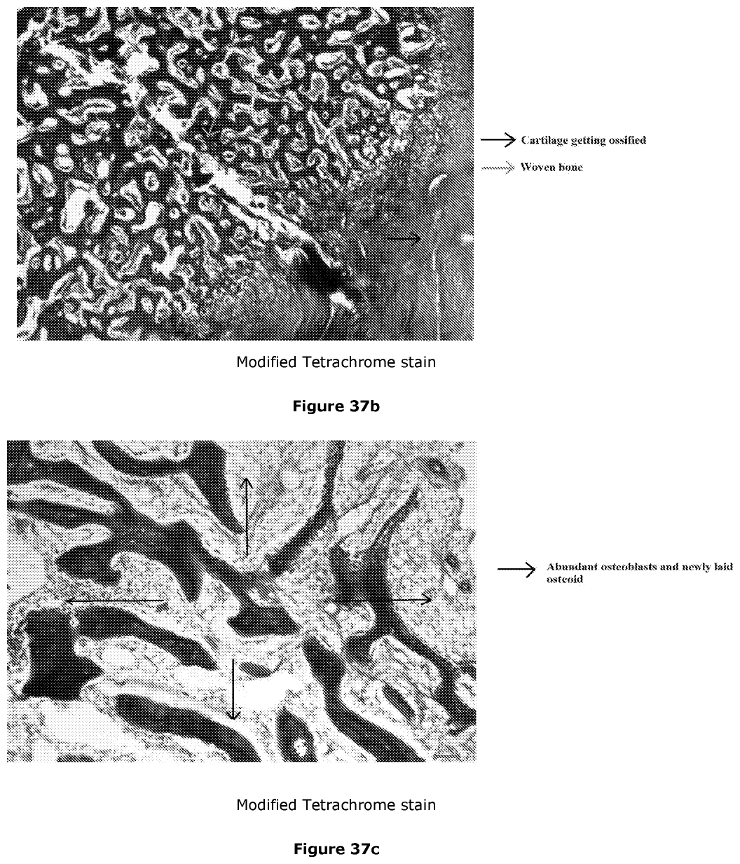

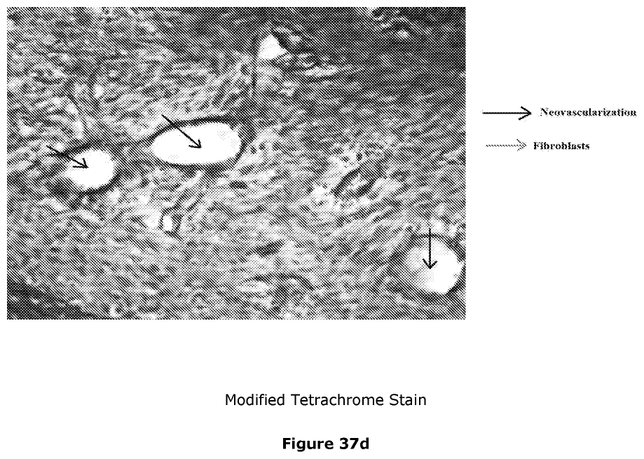

The procedure adapted has been serially shown in the photographs (FIG. 32). Though the limb got fractured it did not receive any specific treatment for it. Still the 9^{th} day there was a scarce response to heal but by the 16^{th} day it had soundly united. (FIG. 33). The xray of the specimen after dissection showed the extension of the callus almost over the entire femur. (FIG. 34). The HPE was specifically focused towards the tissue between the fractured ends where the granules had been packed (FIG. 35). The significant observations were a) Nearly the whole of the granules had resorbed except occasional trace of it. b) abundant cartilage had formed between the ends indicating the enchondral ossification. c) woven bone formed inbetween the ends of the fracture was a proof of the rapidity of the fusion occuring d) the absence of multinucleated giant cells indicate the bio compatibility of the composite. e) similar features were observed in both the staining (FIG. 36). The modified tetrachrome staining throws much more information than the above two. a) The new lamellar bone formed in continuity with the resorbing composite granule b) the sound union by the woven bone formed from chondral ossification c) the abundance of osteoblasts and the osteoid d) exuberent neo vascularisation among the fibroblasts are well seen (FIG. 37-a-b-c-d)

### Example 9: In Vivo Evaluation of the Composite Strips

The in vivo studies were conducted with the Ethics committee approval (Ethical committee approval no ABS/IAEC/18Oct. 2019/003-)

Three male rabbits were procured and domesticated in the same way as explained before. AgFP/ZnFP/MgFP composites were made with PLA+PDLLA+PPF by Gelfoam casting under rapid heating. They were of 1 mm thickness and cut into size of 2*20 mm. The cut specimens were sterilised by Ethylene oxide gas sterilisation.

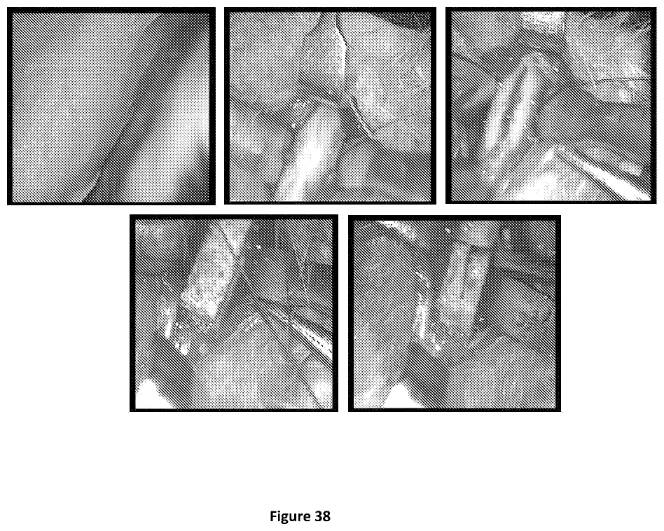

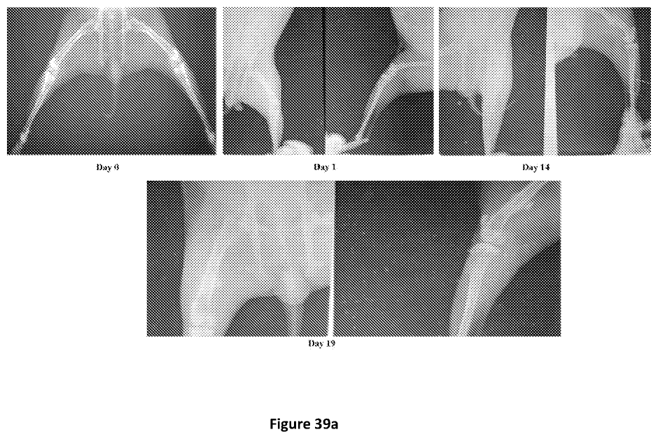

The animals were anaesthetised, limb prepared and femur exposed as described in the previous study. Narrow cuts were made with no701 conical dental burr at an angle of 45° to the femur to make it extremely thin cut 0.3-0 vicryl was threaded around the femur and both the ends were kept free. Two layers of the 2*20 mm sterilised composite was kept over the cut made allowing the marrow blood to choke the specimen. The vicryl was tied around the specimen so that the specimen does not slip or move away and the wound was closed in layers (FIG. 38). The procedure was done for all the three specimens one on each animal

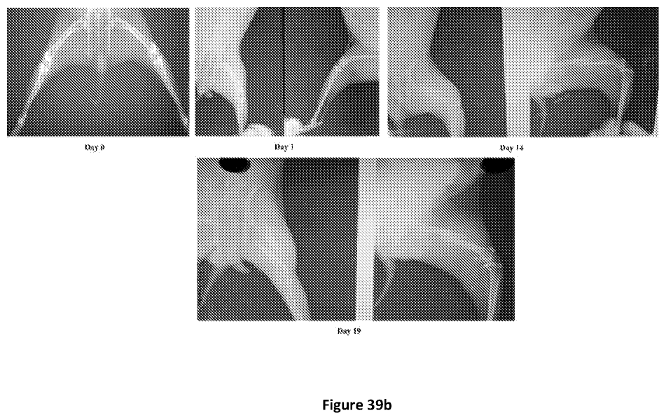

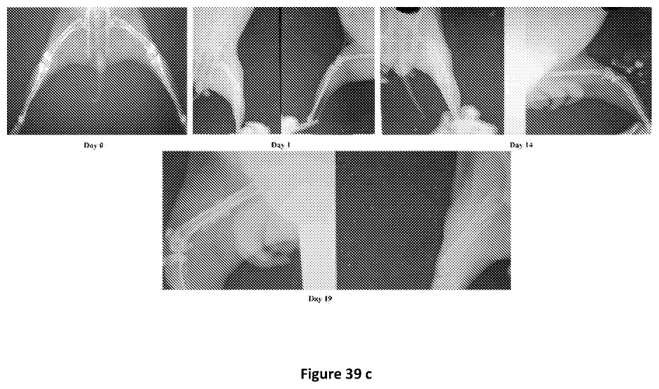

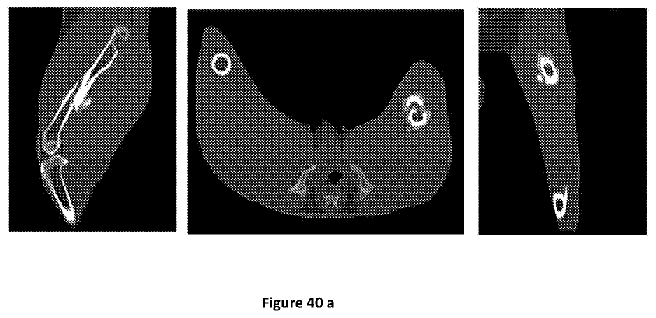

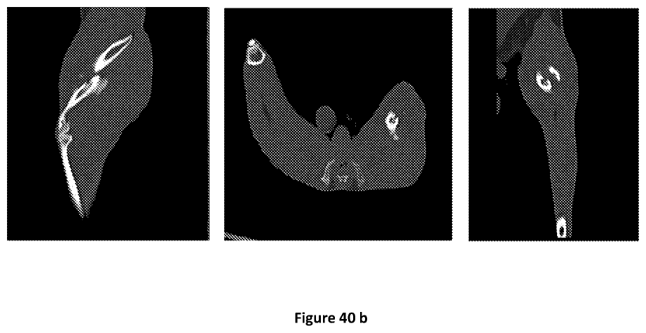

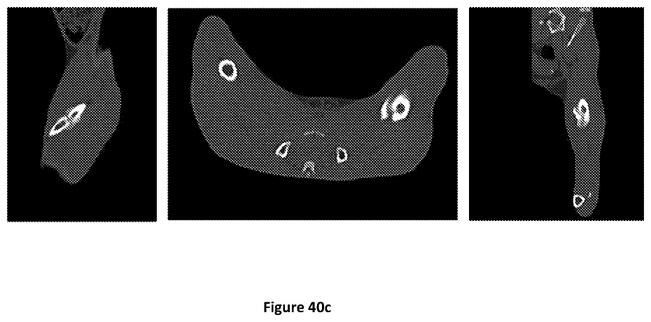

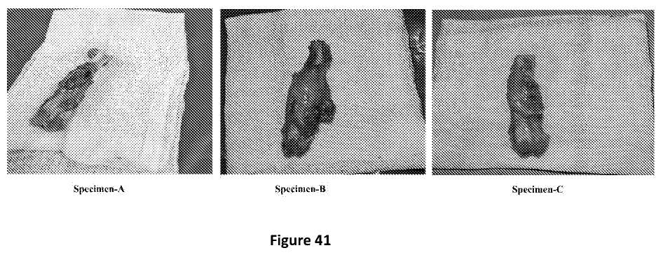

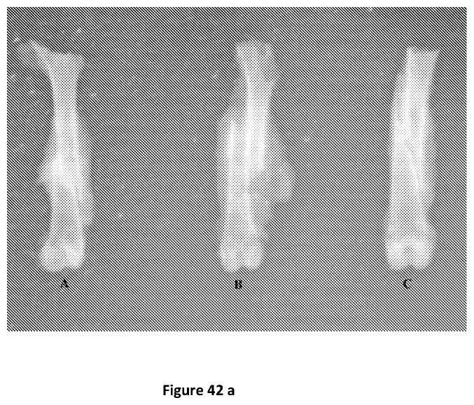

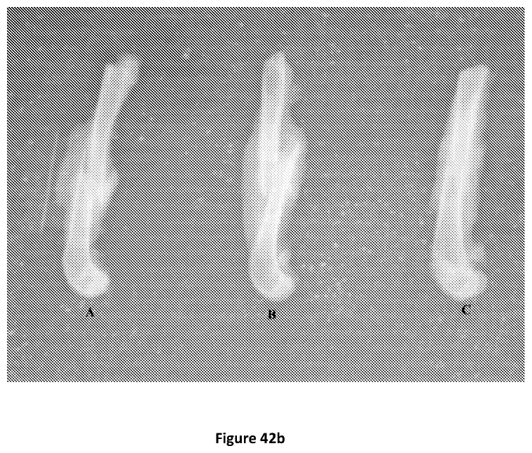

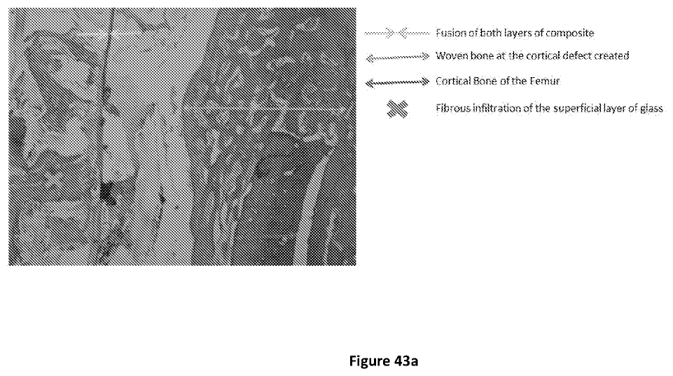

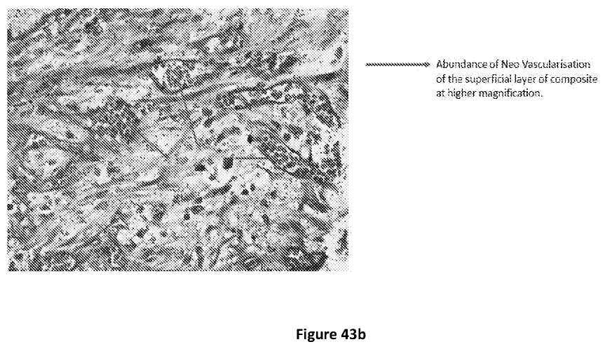

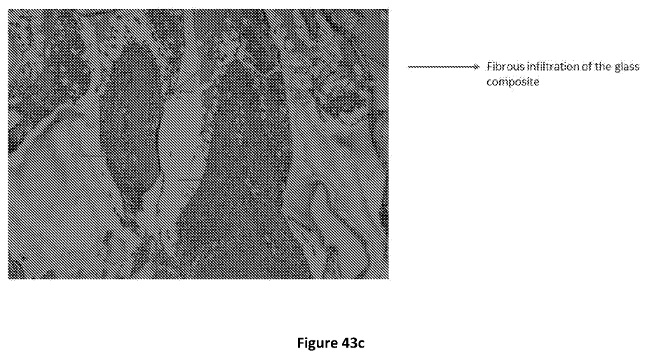

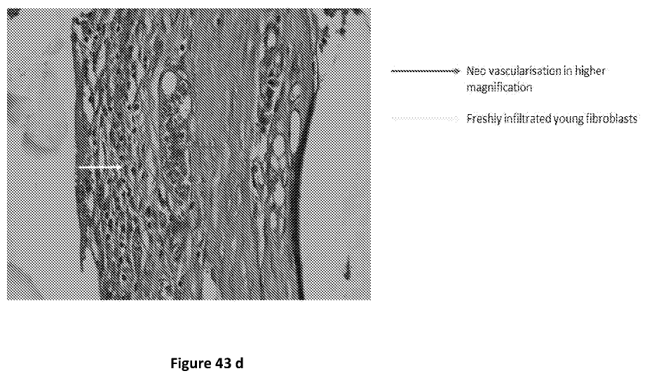

The animals were cared for in the post-operative period with nourishing food. The day one x-ray didn't show the specimen in either view as the specimens were translucent to the x-ray. X-ray evaluation was done under sedation on the 1^{st}, 9^{th} and 16^{th} day. (FIG. 39a,39b,39c) Clinical union occurred as early as the 15^{th} day. CT evaluation was done on the 19^{th} day. (FIG. 40, 40a, 40b) The animals were euthanized as per the protocol and the limb harvested, denuded of skin and muscles and bone preserved in 10% formalin (FIG. 41). X-Rays of the specimens taken (FIG. 42,42a) and then sent for histo pathological evaluation in both EH stain and Eosin stain (FIG. 43,a,b,c,d.)

The procedure adapted is shown in the serial photographs in the FIG. 38, where two layers of 1 mm thick strips have been placed over a very narrow corticotomy wound in the shaft of femur and has been retained in position by a single 3-0 vicryl encircling knot. FIG. 39a, b, c, shows no evidence of the placed composite sheet or the corticotomy made as the composite is not radio opaque and the furrow is very narrow. But the x-rays taken on the 9^{th} day showed all three animals had fractured their femur. No specific treatment like immobilisation or interference was done for the fracture. Clical union occurred as early as 15^{th} day, and was confirmed by x-ray on 16^{th} day and CT scan on 19^{th} day. (FIG. 40a,b,c). The harvested limb after euthanizing the animal showed the composite strip was adherent to the bone underneath (FIG. 41). The X-ray of the specimens showed abundant callus along the fracture (FIG. 42) and the composite strip was not seen in the X-ray.

The histo pathological evaluation showed the following features (FIG. 43a) a) Both the layers of the scaffold had merged into one layer b) The composite had attached to the bone beneath. c) There was abundant woven bone formed beneath the composite strip at the level of the corticotomy. (FIG. 43 b) d) The second layer of the composite srip kept away from the corticotomy had profuse infiltration of fibrocytes. (FIG. 43 c,d) e) The fibrous changeover in the superficial layer of the composite had abundant neovascularisation These changes confirm the osteo induction potential of the composite, the ability of the composite to go for bioconversion and high bioactivity of the composite.

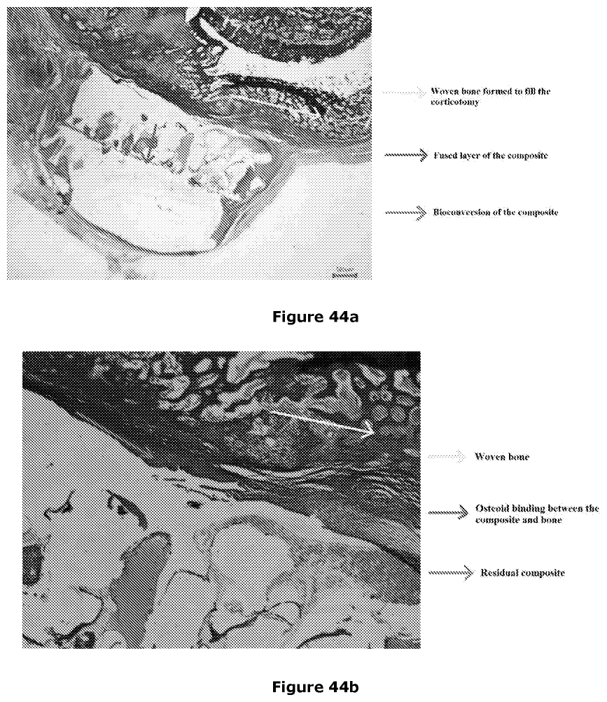

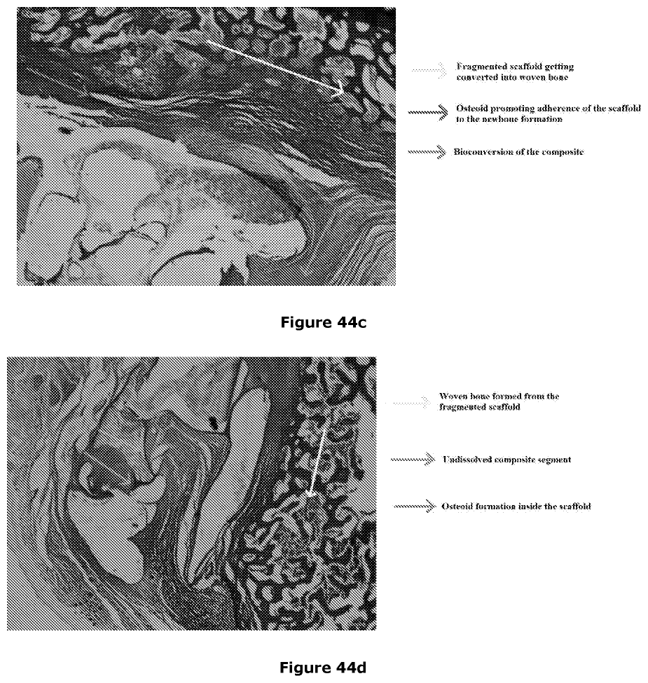

The modified Tetrachrome staining of the specimens with the cross section at the level of the composite confirmed the findings by EH stain and showed the additional features. FIG. 44a shows conversion of the fragmented composite forming woven bone to heal the corticotomy made and the binding of the two layers of the composite strip and random infiltration of the layer close to the bone with fibroblasts and specks of osteiod. On Higher magnification (44b) the fusion of the composite strip to the underlying bone by osteoid is well seen. On further magnification (44 c) the infiltration of the composite by newly formed layers of osteoid are well made out replacing the dissolved area of the composite. FIG. 44d shows the adhesion of the composite strip, the composite strip dissolving and disintegrating to form new woven bone healing the corticotomy, the phenomenal laying of new osteoid in the dissolved portion of the composite.

### Example 10: Preparation of the Scaffold by 3 D Printing

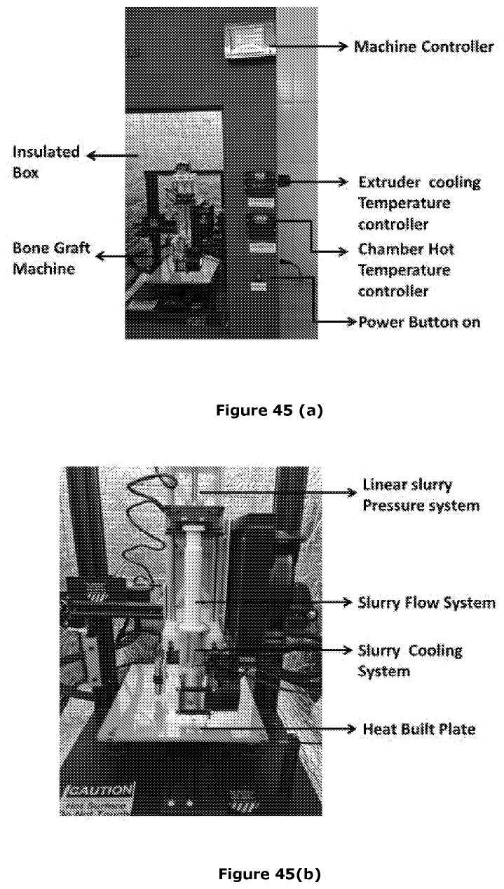

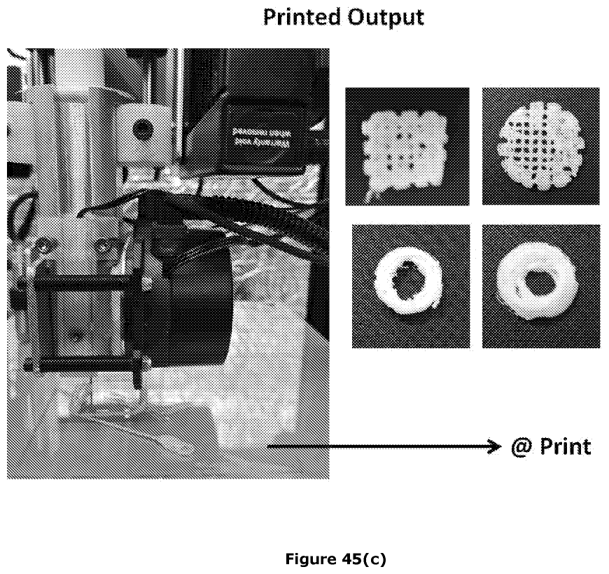

Fused filament fabrication (F1-1-) 3D printer is generally used for fusing plastics, extruded at a higher temperature and cooled to room temperature to build the 3 D model into a product. A customised 3D printer was manufactured for fabricating the composite. The ink printer is maintained at a cool temperature in the printer so that the homogeneity obtained between the components of the composite is not lost. (FIG. 45b). For that purpose, a special cooling chamber was designed. It cools the slurry extruder at 15-20° C. The slurry is extruded to a plate built to get heated upto 100° C. and the chamber temperature of 3040° C. is maintained (FIG. 45a). The extrusion was controlled by conventional CAD software and the required designs were printed (FIG. 45c).

### Advantages

The composite can be made as granules or powders or their mixture which can be used as a filler for bone voids arising out of lesions, infections, tumours of bone which will get converted to bone in a shorter period avoiding amputations and also reduce the morbidity by reducing the time taken for bioconversion.

The composite made as strips can be used as an only graft like that of a Phemister graft which is the commonest type of autogenous graft used by the orthopaedic surgeon. This will reduce the morbidity of the surgery and avoid a second incision to harvest the autograft.

The composites as cylindrical grafts can be used as an interposition graft and can save many long bones with critical sized defects arising out of trauma or other lesions.

The composites can be custom made to a graft by rapid prototyping method so that a specific portion of a bone can be replaced when diseased rather than being amputated.

The Synthetic Composite of the Invention has Following Characters

a) Biocompatible; b) Bioactive; c) Biodegradable; d) Nontoxic to the recipient; e) Bioconductive; f) Bioinductive; g) Bioconvertible; h) Rate of degradation to match the rate of bio-conversion; i) sterilisable; j) easy to be produced in bulk; k) workable to the desired shape; 1) cost effective.

1. A synthetic composite for a bone graft comprising: bio inert polymers comprising poly lactic acid, poly D, L-Lactic acid; bio active polymer consisting of polypropylene fumarate or a diester of fumaric acid and propylene diol (1,2-Diol); and a bioactive inorganic component consisting of a metal fluorophosphates glass powder wherein the bioactive inorganic metal fluorophosphates glass powder is up to 30% (w/w) of the composite.

2. The composite as claimed in claim 1, wherein the metal fluorophosphate glass is selected from one of zinc fluorophosphate, magnesium fluorophosphate or silver fluorophosphate.

3. The composite as claimed in claim 1, wherein the polylactic acid is in the range of 54% (w/w) to 68% (w/w).

4. The composite as claimed in claim 1, wherein the 1,2 diol is in the range of 3% (w/w) to 10% (w/w).

5. The composite as claimed in claim 1, wherein the poly D, L-lactic acid is in the range of 10% (w/w) to 28% (w/w).

6. The composite as claimed in claim 1, wherein the polypropylene fumarate is in the range of 3% (w/w) to 10% (w/w).

7. The composite as claimed in claim 1, wherein the metal fluorophosphate in the composite is in the range of 10% (w/w) to 30% (w/w).

8. The composite as claimed in claim 1, comprising polylactic acid, 1,2 diol, and zinc fluorophosphate.

9. The composite as claimed in claim 1, comprising polylactic acid, poly D, L-Lactic acid, 1,2 diol and zinc fluorophosphate.

10. The composite as claimed in claim 1, comprising polylactic acid, poly propylene fumarate and zinc fluorophosphate.

11. The composite as claimed in claim 1, comprising polylactic acid, poly D, L-Lactic acid, poly propylene fumarate and zinc fluorophosphate.

12. The composite as claimed in claim 1, comprising polylactic acid, 1, 2 diol and magnesium fluorophosphate.

13. The composite as claimed in claim 1, comprising polylactic acid, poly D, L-Lactic acid, 1,2 diol and magnesium fluorophosphate.

14. The composite as claimed in claim 1, comprising polylactic acid, poly propoylene fumarate and magnesium fluorophosphate.

15. The composite as claimed in claim 1, comprising polylactic acid, poly D, L-lactic acid, poly propylene fumarate and magnesium fluorophosphate.

16. The composite as claimed in claim 1, comprising polylactic acid, 1, 2 diol and silver fluorophosphate.

17. The composite as claimed in claim 1, comprising polylactic acid, poly D, L-Lactic acid, 1,2 diol and silver fluorophosphate.

18. The composite as claimed in claim 1, comprising polylactic acid, poly propylene fumarate and silver fluorophosphate.

19. The composite as claimed in claim 1, comprising polylactic acid, poly D, L-lactic acid, poly propylene fumarate and silver fluorophosphate.

20. The composite as claimed in claim 1, is one of a powder, or a scaffold, optionally wherein the scaffold is a strip or a cylinder or a tube and the like.

21. (canceled)

22. A method of preparing a synthetic composite as claimed in claim 20, comprising the steps of:
mixing the composite of claim 1 in a solvent with the magnetic stirrer or sonicated to obtain a homogenous mixture;
casting the mixture over hot glass plate and bringing the solution to boil;
evaporation of the solvent by continuous boiling; and
obtaining an interconnected porous scaffold with the homogenous distribution of the components of the composite.

23. The method as claimed in claim 22, wherein;
(a) the solvent is one of dichloromethane, acetone, toluene or chloroform; and/or
(b) the porosity of the scaffold ranges from 20%-40%.

24. (canceled)

25. The composite as claimed in claim 1, wherein the scaffold is made by a custom made 3D printer such as herein described by the direct ink printing technique comprising the following steps:
homogenisation of the components and cooling the same to 10 to 30 degrees;
loading of the ink into a pressure-controlled extruder which is non adhesive to the ink;
programming of the shape, thickness, porosity, and layers required is fed by a computer;
obtaining the desired scaffold.
